(19) United States
(12) Patent Application Publication
(10) Pub. No.: US 2026/0026701 A1
(43) Pub. Date: Jan. 29, 2026

(54) BLOOD FLOW MEASUREMENT DEVICE

(71) Applicant: FUJIFILM Corporation, Tokyo (JP)

(72) Inventors: Yukito SAITOH, Minamiashigara-shi (JP); Mayumi NOJIRI, Minamiashigara-shi (JP); Ryuji SANETO, Minamiashigara-shi (JP)

(73) Assignee: FUJIFILM Corporation, Tokyo (JP)

(21) Appl. No.: 19/181,453

(22) Filed: Apr. 17, 2025

Related U.S. Application Data
(63) Continuation of application No. PCT/JP2023/037840, filed on Oct. 19, 2023.

(30) Foreign Application Priority Data
Oct. 20, 2022 (JP) 2022-168281

(51) Int. Cl. A61B 5/026

(52) U.S. Cl. CPC A61B 5/0261; A61B 2562/0233

(57) ABSTRACT

Provided is a blood flow measurement device having excellent measurement accuracy. The blood flow measurement device includes a light source unit that irradiates an object with near-infrared rays, and a light receiving section that receives scattered light generated by scattering of the near-infrared rays emitted from the light source unit by the object, the blood flow measurement device further including a first polarizing element that is disposed on a front surface of the light source unit, includes a layer formed of a liquid crystal compound, and changes a polarization state of the near-infrared rays, and a second polarizing element that is disposed on a front surface of the light receiving section, includes a layer formed of a liquid crystal compound, and changes a polarization state of the near-infrared rays.

### CROSS-REFERENCE TO RELATED APPLICATIONS

This application is a Continuation of PCT International Application No. PCT/JP2023/037840 filed on Oct. 19, 2023, which claims priority under 35 U.S.C. § 119 (a) to Japanese Patent Application No. 2022-168281 filed on Oct. 20, 2022. The above applications are hereby expressly incorporated by reference, in their entirety, into the present application.

### BACKGROUND OF THE INVENTION

### 1. Field of the Invention

The present invention relates to a blood flow measurement device.

### 2. Description of the Related Art

It is known that the measurement of blood flow rates in the brain, the muscle, the organ, and the like of a living body is applied to diagnosis of a physical function, health management, information mediation between a human body and a device, and the like. In particular, with regard to the brain, there has been provided a device that acquires information indicating an activity state of the brain by providing a near-infrared ray irradiation unit and a near-infrared ray detection unit in a cerebral blood flow rate measurement device called a headset, detecting a change in blood flow rate of a brain surface, and processing the detected data with a data processing device.

For example, JP2020-054649A describes a blood flow rate measurement device including a first body part, a second body part, and a hinge, in which the first body part has a first housing including a first bottom surface, a light source that irradiates a portion other than the first housing from the first bottom part with near-infrared rays, and a first light receiving section that receives near-infrared rays from a first bottom surface side outside the first housing, the second body part has a second housing including a second bottom surface and a second light receiving section that receives near-infrared rays from a second bottom surface side outside the second housing, and the hinge bonds the first body part with the second body part by variably forming an angle formed between the first bottom surface and the second bottom surface.

### SUMMARY OF THE INVENTION

Such a blood flow measurement device obtains information on a blood flow rate by detecting near-infrared rays that are scattered while being partially absorbed by the blood vessel (blood). Since the near-infrared rays irradiated for the measurement are scattered, the detected near-infrared rays are weak. In addition, since the irradiated near-infrared rays are reflected on a portion other than a measurement portion, such as a surface of a human body, the near-infrared rays reflected on a portion other than the measurement portion are detected as a noise component. Blood flow measurement devices in the related art had problems in that the SN ratio is low and the measurement accuracy is poor since they had difficulty in distinguishing near-infrared rays of a detection target and near-infrared rays of a noise component.

Δn object of the present invention is to solve such problems of the related art and to provide a blood flow measurement device having excellent measurement accuracy.

In order to accomplish the object, the present invention has the following configurations.

[1] A blood flow measurement device including:
a light source unit that irradiates an object with near-infrared rays; and
a light receiving section that receives scattered light generated by scattering of the near-infrared rays emitted from the light source unit by the object, the blood flow measurement device further including:
a first polarizing element that is disposed on a front surface of the light source unit, includes a layer formed of a liquid crystal compound, and changes a polarization state of the near-infrared rays; and
a second polarizing element that is disposed on a front surface of the light receiving section, includes a layer formed of a liquid crystal compound, and changes a polarization state of the near-infrared rays.

[2] The blood flow measurement device according to [1],
in which the layer formed of the liquid crystal compound included in the first polarizing element is a linear polarizer.

[3] The blood flow measurement device according to [2], in which the first polarizing element further includes a λ/4 plate.

[4] The blood flow measurement device according to [3], in which the λ/4 plate exhibits reverse wavelength dispersibility.

[5] The blood flow measurement device according to [1],
in which the first polarizing element has a first linear polarizer, a retardation layer, and a second linear polarizer in this order, and
at least one of the first linear polarizer or the second linear polarizer is the layer formed of the liquid crystal compound.

[6] The blood flow measurement device according to [5],
in which a retardation layer exhibits reverse wavelength dispersibility.

[7] The blood flow measurement device according to any one of [1] to [6],
in which the layer formed of the liquid crystal compound included in the first polarizing element has a liquid crystal alignment pattern in which an orientation of an optical axis derived from the liquid crystal compound changes while continuously rotating along at least one in-plane direction.

[8] The blood flow measurement device according to any one of [1] to [7],
in which the liquid crystal compound is a rod-like liquid crystal compound or a disk-like liquid crystal compound.

According to the present invention, it is possible to provide a blood flow measurement device having excellent measurement accuracy.

### BRIEF DESCRIPTION OF THE DRAWINGS

FIG. 1 is a view conceptually showing an example of a blood flow measurement device of an embodiment of the present invention.

FIG. 2 is a conceptual view showing a part of an example of the blood flow measurement device of the embodiment of the present invention.

FIG. 3 is a conceptual view showing a part of another example of the blood flow measurement device of the embodiment of the present invention.

FIG. 4 is a conceptual view showing a part of another example of the blood flow measurement device of the embodiment of the present invention.

FIG. 5 is a conceptual view showing a part of another example of the blood flow measurement device of the embodiment of the present invention.

FIG. 6 is a view conceptually showing a liquid crystal diffraction element included in a first polarizing element of the blood flow measurement device shown in FIG. 5.

FIG. 7 is a plan view showing the liquid crystal diffraction element shown in FIG. 6.

FIG. 8 is a conceptual view showing an action of the liquid crystal diffraction element shown in FIG. 6.

FIG. 9 is a conceptual view showing an action of the liquid crystal diffraction element shown in FIG. 8.

FIG. 10 is a view schematically showing an example of an exposure device that exposes an alignment film of the liquid crystal diffraction element shown in FIG. 6.

FIG. 11 is a view conceptually showing another example of the liquid crystal diffraction element.

FIG. 12 is a view conceptually showing another example of the liquid crystal diffraction element.

### DESCRIPTION OF THE PREFERRED EMBODIMENTS

Hereinafter, a blood flow measurement device of an embodiment of the present invention will be described in detail based on suitable embodiments shown in the accompanying drawings.

In the present specification, numerical ranges represented by “to” include numerical values before and after “to” as lower limit values and upper limit values.

### [Blood Flow Measurement Device]

The blood flow measurement device of the embodiment of the present invention is a blood flow measurement device including:
a light source unit that irradiates an object with near-infrared rays and a light receiving section that receives scattered light generated by scattering of the near-infrared rays emitted from the light source unit by the object, the blood flow measurement device further including:
a first polarizing element that is disposed on a front surface of the light source unit, includes a layer formed of a liquid crystal compound, and changes a polarization state of the near-infrared rays; and
a second polarizing element that is disposed on a front surface of the light receiving section, includes a layer formed of a liquid crystal compound, and changes a polarization state of the near-infrared rays.

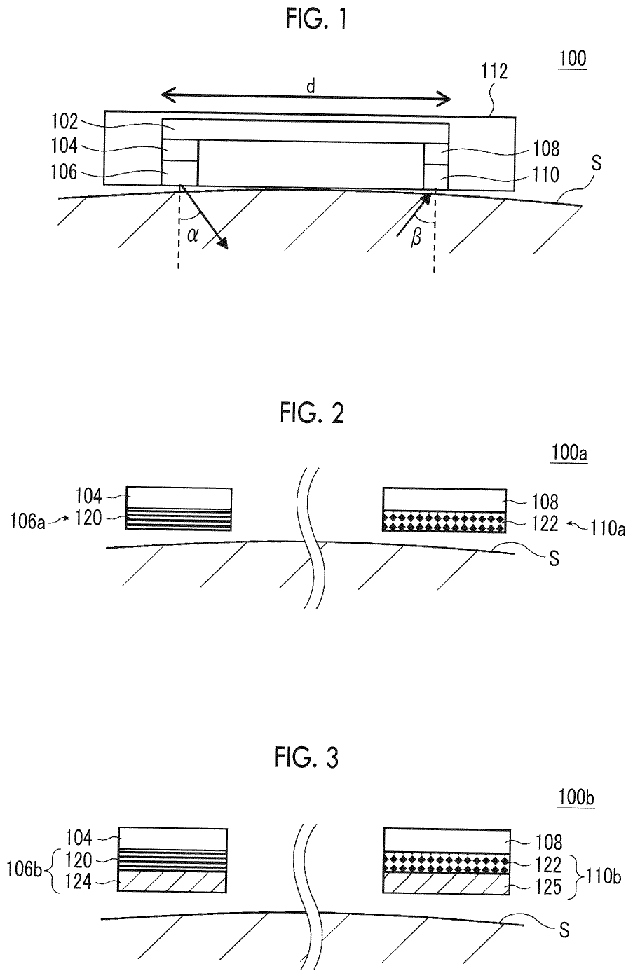

FIG. 1 conceptually shows an example of the blood flow measurement device of the embodiment of the present invention.

A blood flow measurement device 100 shown in FIG. 1 is a device that acquires information related to a blood flow by irradiating a living body with near-infrared rays and detecting the near-infrared rays reflected on the living body.

The blood flow measurement device 100 shown in FIG. 1 has a control unit 102, a light source unit 104, a first polarizing element 106, a light receiving section 108, a second polarizing element 110, and a housing 112.

### <Control Unit>

The control unit 102 functions as a support substrate that supports the light source unit 104 and the first polarizing element 106, and the light receiving section 108 and the second polarizing element 110, and performs measurement control and data processing in the blood flow measurement device 100. That is, the control unit 102 controls the irradiation timing, the light amount, and the like of the near-infrared rays by the light source unit 104, and performs various types of processing on the data obtained by the light reception by the light receiving section 108 to calculate the amount of change in the blood flow, the pulse rate, and the like. The pulse rate corresponds to the heart rate.

The control unit 102 has, for example, a processor such as a central processing unit (CPU) or digital signal processor (DSP), and a memory, and executes processing by a computer program, firmware, or the like that is developed on the memory in an executable manner. The control unit 102 may be a dedicated hardware circuit, a field programmable gate array (FPGA), or the like that activates the light source unit 104 and the light receiving section 108, and executes the cooperation processing with each constituent.

As shown in FIG. 1, in the control unit 102, the light source unit 104 and the light receiving section 108 are disposed to be spaced with a predetermined distance d in the plane direction of a surface of the control unit 102.

Furthermore, in the example shown in the drawing, the control unit 102 is configured to also function as a support substrate that supports the light source unit 104 and the first polarizing element 106, and the light receiving section 108 and the second polarizing element 110, but the present invention is not limited thereto. The support substrate that supports the light source unit 104 and the first polarizing element 106, and the light receiving section 108 and the second polarizing element 110 and the control unit 102 may be separate members.

### <Light Source Unit>

The light source unit 104 is used for irradiating a living body S with near-infrared rays. The light source unit 104 includes a near-infrared ray source that irradiates near-infrared rays. It is preferable that the near-infrared rays irradiated by the light source unit 104 have a wavelength of 650 nm to 1,400 nm.

As the near-infrared ray source, for example, light emitting diodes (LEDs) or laser diodes (LDs) can be used.

The light source unit 104 basically emits unpolarized near-infrared rays. Furthermore, in a case where the near-infrared ray source has a linear polarizer and emits linearly polarized near-infrared rays, the linear polarizer is regarded as the linear polarizer included in the first polarizing element in the present invention.

In addition, the light source unit 104 may be one that irradiates two or more types of near-infrared rays having different wavelengths. For example, the light source unit 104 may be a unit that irradiates near-infrared rays having wavelengths of 780 nm and 830 nm. Such a light source unit 104 may be configured to have a plurality of light sources that irradiate near-infrared rays having different wavelengths, or may be configured to irradiate near-infrared rays having different wavelengths by combining a light source that irradiates near-infrared rays having a wide wavelength range, a filter that transmits a specific wavelength range, and the like.

### <Light Receiving Section>

The light receiving section 108 is a section that receives (detects) the near-infrared rays reflected in the body of the living body S.

The light receiving section 108 includes, for example, a photoelectric conversion element such as a photodiode and a phototransistor, which outputs a current in accordance with the amount of received near-infrared rays, an amplification circuit that amplifies the output current of the photoelectric conversion element, and an analog-to-digital (AD) converter.

The light receiving section 108 converts the received light into a voltage signal and outputs the voltage signal as a light detection signal.

Furthermore, the size of the light receiving section 108 is not limited as long as the near-infrared rays reflected in the body of the living body S can be received (detected), but it is preferable that the area is increased and the incidence angle is increased so that high detection sensitivity can be obtained.

In addition, in a case where the light source unit 104 emits two or more kinds of near-infrared rays having different wavelengths, it is preferable that the light receiving section 108 receives (detects) near-infrared rays for each wavelength. In this case, the light receiving section 108 may be configured to have a combination of a filter that transmits one wavelength range and cuts the other wavelength range and a photoelectric conversion element, and a combination of a filter that transmits the other wavelength range and cuts the one wavelength range and a photoelectric conversion element.

### <First Polarizing Element>

The first polarizing element 106 is an element that is disposed on the front surface of the light source unit 104, that is, the irradiation surface, and that changes a polarization state of near-infrared rays emitted from the light source unit 104. The first polarizing element 106 includes a layer formed of a liquid crystal compound.

The first polarizing element 106 converts the polarization state of the near-infrared rays emitted from the light source unit 104 into linearly polarized light or circularly polarized light having a desired polarization state and causes the linearly polarized light or circularly polarized light to be incident into the inside of the living body S.

The configuration of the first polarizing element 106 will be described in detail later.

### <Second Polarizing Element>

The second polarizing element 110 is an element that is disposed on the front surface of the light receiving section 108, that is, on the light receiving surface, that scatters near-infrared rays in the body of the living body S, and changes a polarization state of the near-infrared rays incident on the light receiving section 108. The second polarizing element 110 includes a layer formed of a liquid crystal compound.

The second polarizing element 110 converts the polarization state of the near-infrared rays scattered in the body of the living body S into linearly polarized light or circularly polarized light having a certain polarization state, and causes the linearly polarized light or circularly polarized light to be incident on the light receiving section 108.

The configuration of the second polarizing element 110 will be described in detail later.

The blood flow measurement device 100 may have, in addition to the above-described components, a housing 112 that houses each of the components, a holding mechanism such as a band for mounting the device on the head, the arm, the leg, or the like of a user (living body S), and the like. The blood flow measurement device 100 is mounted on the head, the arm, the leg, or the like of the user (living body S) by a holding mechanism such that the irradiation surface faces toward the living body S side and the light receiving surface faces toward the living body S side such that the near-infrared rays from the light source unit 104 are irradiated into the living body S and the near-infrared rays scattered in the living body S is received by the light receiving section 108.

Δn action of such a blood flow measurement device 100 will be described.

The blood flow measurement device 100 worn on the head, the arm, the leg, or the like of the living body S irradiates near-infrared rays from the light source unit 104. The near-infrared rays irradiated from the light source unit 104 are incident on the first polarizing element 106, and the polarization state thereof is changed by the first polarizing element 106 to be incident into the inside of the living body S. The irradiated near-infrared rays are partially absorbed and scattered, for example, in the vicinity of the cerebral cortex of the brain and the vicinity of blood vessels of the arm. A part of the scattered near-infrared rays travels toward the light receiving section 108 side and is incident on the second polarizing element 110. The second polarizing element 110 changes a polarization state of the incident near-infrared rays and causes the near-infrared rays to be incident on the light receiving section 108. The light receiving section 108 receives near-infrared rays, converts the near-infrared rays into an electric signal, and outputs the electric signal. The electric signal (data) output from the light receiving section 108 is transmitted to the control unit 102. The control unit 102 performs various types of processing on the received data to calculate a blood flow change amount, a pulse rate, and the like.

Here, for example, in the cerebral cortex of the brain, the blood flow rate changes in accordance with the activity state of the brain. As a result, the amount of oxygenated hemoglobin and the amount of non-oxygenated hemoglobin in the blood in the cerebral cortex change in accordance with the activity state of the brain. In addition, the absorption characteristics or the scattering characteristics of the near-infrared rays in the vicinity of the cerebral cortex change due to a change in the amount of hemoglobin, a change in the amount of oxygen, or the like. Therefore, the amount of near-infrared rays received by the light receiving section 108 changes. Accordingly, the control unit 102 can obtain information on the blood flow (the amount of change in the blood flow, the pulse rate, and the like) in the vicinity of the cerebral cortex or the like from the data on the amount of near-infrared rays received by the light receiving section 108.

In addition, the change in the absorption characteristics or the scattering characteristics of the near-infrared rays caused by the change in the amount of hemoglobin, the change in the amount of oxygen, or the like vary depending on the wavelength. Therefore, for example, in the cerebral cortex of the brain, the change in the amount of near-infrared rays received by the light receiving section 108 varies depending on the wavelength according to the activity state of the brain. That is, the ratio of the amount of light for each wavelength received by the light receiving section 108 changes in accordance with the activity state of the brain. Accordingly, by configuring the light source unit 104 to irradiate two or more kinds of near-infrared rays having wavelengths that are different from each other such that the light receiving section 108 is configured to receive light for each wavelength to obtain data on the amount of light for each wavelength, it is possible to obtain information on the blood flow (amount of change in blood flow, pulse rate, and the like) from the data on the ratio of the amounts of light received at two (or three or more) wavelengths.

Here, in a blood flow measurement device that irradiates the inside of a living body with near-infrared rays and receives the near-infrared rays scattered in the vicinity of a blood vessel to acquire information on a blood flow, the scattered near-infrared rays are received. Therefore, the amount of the near-infrared rays received by the light receiving section is about 1/100 to 1/1,000 of the amount of the irradiated near-infrared rays, which is weak. In addition, the near-infrared rays irradiated from the light source unit are reflected on a portion other than a measurement portion, such as a surface of a human body and a surface of an organ. In a case where the near-infrared rays reflected on a portion other than such a measurement portion are received by the light receiving section, the near-infrared rays serve as an unnecessary noise component. In a blood flow measurement device in the related art, it is difficult to distinguish between the near-infrared rays of a detection target and the near-infrared rays of a noise component, and therefore, there is a problem that the SN ratio is low and the measurement accuracy is poor.

In contrast, the blood flow measurement device 100 of the embodiment of the present invention has the first polarizing element 106 that includes a layer formed of a liquid crystal compound on the front surface of the light source unit 104 and changes a polarization state of near-infrared rays, and has the second polarizing element 110 that includes a layer formed of a liquid crystal compound on the front surface of the light receiving section 108 and changes a polarization state of near-infrared rays.

The first polarizing element 106 converts the near-infrared rays emitted from the light source unit 104 into predetermined linearly polarized light or circularly polarized light. A part of the polarized light changed by the first polarizing element 106 is incident into the living body S and is scattered in the vicinity of the blood vessel. At this time, the near-infrared rays to be measured are depolarized by scattering, and therefore, the near-infrared rays are in a polarization state different from a predetermined polarization state, for example, unpolarized light. A part of the scattered near-infrared rays of the measurement target is incident on the second polarizing element 110. The second polarizing element 110 converts, for example, the near-infrared rays to be measured, which are unpolarized light, into predetermined linearly polarized light or circularly polarized light. The near-infrared rays to be measured, which are the polarized light changed by the second polarizing element 110, are received by the light receiving section 108.

On the other hand, a part of the polarized light changed to a predetermined polarization state by the first polarizing element 106 is reflected on a portion other than a measurement portion, such as a surface of a human body and an interface of an organ. Since the polarized light is not eliminated by reflection, the polarized light reflected on a portion other than the measurement portion is in a certain polarization state. In a case where the near-infrared rays reflected on a portion other than the measurement portion travel toward the light receiving section 108, the near-infrared rays are incident on the second polarizing element 110 disposed on the front surface of the light receiving section 108. As described later, since the second polarizing element 110 has a configuration in which the polarized light reflected on a portion other than the measurement portion, that is, the near-infrared rays as a noise component are cut, the amount of light received by the light receiving section 108 can be reduced.

As described above, in the blood flow measurement device 100 of the embodiment of the present invention, the near-infrared rays of the detection target and the near-infrared rays of the noise component can be distinguished from each other and the noise component can be cut, and therefore, the SN ratio can be improved and the measurement accuracy can be improved.

Here, in the blood flow measurement device 100 of the embodiment of the present invention, the first polarizing element 106 and the second polarizing element 110 include a layer formed of a liquid crystal compound. In the first polarizing element 106 and the second polarizing element 110, the layer formed of the liquid crystal compound is a layer for changing the polarization state of the incident near-infrared rays. Specifically, as described later, the layer formed of the liquid crystal compound is a linear polarizer or a liquid crystal diffraction element. The layer formed of the liquid crystal compound can be a layer that changes a polarization state with high efficiency with respect to near-infrared rays. In addition, in the case of a linear polarizer, an absorption-type linear polarizer that does not reflect near-infrared rays can be used, and therefore, the occurrence of reflected light that can serve as a noise can be suppressed.

Accordingly, in the blood flow measurement device 100 of the embodiment of the present invention, by configuring the first polarizing element 106 and the second polarizing element 110 to include a layer formed of a liquid crystal compound, the action of cutting the noise component by shielding the reflected light of the near-infrared rays, of which the polarization state is changed by the first polarizing element 106, described above, by the second polarizing element 110 can be appropriately exhibited.

Furthermore, the distance d from the light source unit 104 to the light receiving section 108 is not particularly limited. Since a depth from the surface of the living body S at which the information on the blood flow is obtained changes in accordance with the distance d, the distance d may be set depending on the depth at which the information on the blood flow is desired to be obtained.

Hereinafter, the configurations of the first polarizing element 106 and the second polarizing element 110 will be described.

FIG. 2 is a conceptual view showing a part of an example of a blood flow measurement device of the embodiment of the present invention.

A blood flow measurement device 100a shown in FIG. 2 has a light source unit 104, a first polarizing element 106a, a light receiving section 108, and a second polarizing element 110a. Furthermore, in the blood flow measurement device 100a shown in FIG. 2, the control unit, the housing, and the like are not shown. In addition, in the blood flow measurement device 100a, the light source unit 104 and the light receiving section 108 have the same configurations as the light source unit 104 and the light receiving section 108 described in the blood flow measurement device 100 shown in FIG. 1, and thus the description thereof will be omitted. The same applies to FIGS. 3 to 5 which will be described later.

In the blood flow measurement device 100a shown in FIG. 2, the first polarizing element 106a has a linear polarizer 120 as the layer formed of the liquid crystal compound. In addition, in a preferred aspect, the second polarizing element 110a has a linear polarizer 122 as the layer formed of the liquid crystal compound. The linear polarizer 120 contained in the first polarizing element 106a and the linear polarizer 122 contained the second polarizing element 110a are disposed such that transmission axes thereof are substantially orthogonal to each other. For example, in the example shown in FIG. 2, the transmission axis of the linear polarizer 120 contained in the first polarizing element 106a may be set to transmit linearly polarized light that vibrates in the left-and-right direction in the drawing, and the transmission axis of the linear polarizer 122 contained in the second polarizing element 110a may be set to transmit linearly polarized light that vibrates in the direction perpendicular to the paper plane in the drawing.

In such a blood flow measurement device 100a, in a case where near-infrared rays are irradiated from the light source unit 104, the linear polarizer 120 of the first polarizing element 106a changes the near-infrared rays into, for example, linearly polarized light that vibrates in the left-and-right direction in the drawing. The near-infrared rays that have been linearly polarized by the linear polarizer 120 (first polarizing element 106a) are incident into the living body S. The near-infrared rays with which the living body S is irradiated are partially absorbed and scattered in the vicinity of the blood vessel. At this time, the near-infrared rays are depolarized from linearly polarized light and thus serve as unpolarized light. A part of the scattered near-infrared rays travels toward the light receiving section 108 side and is incident on the second polarizing element 110a. The linear polarizer 122 of the second polarizing element 110a transmits the incident near-infrared rays as linearly polarized light that vibrates in the direction perpendicular to the paper plane. The light receiving section 108 receives the linearly polarized near-infrared rays, converts the linearly polarized near-infrared rays into an electrical signal, and outputs the electrical signal to the control unit. The control unit performs various types of processing on the received data to calculate a blood flow change amount, a pulse rate, and the like.

On the other hand, a part of the near-infrared rays changed into linearly polarized light by the linear polarizer 120 (first polarizing element 106a) is reflected on a portion other than the measurement portion, such as a surface of a human body and an interface of an organ. At this time, since the polarization is not eliminated, the linearly polarized light vibrates in the left-and-right direction in the drawing and is incident on the linear polarizer 122 (second polarizing element 110a) as it is. Since the linear polarizer 122 of the second polarizing element 110a has a transmission axis in the direction perpendicular to the paper plane, it absorbs the linearly polarized light that vibrates in the left-and-right direction in the drawing without transmitting the light. This makes it possible to cut the linearly polarized light reflected on a portion other than the measurement portion, that is, the near-infrared rays as a noise component, and makes it possible to suppress the light receiving section 108 from receiving the noise component.

Moreover, as described above, by forming the linear polarizer 120 and the linear polarizer 122 using the liquid crystal compound, the linear polarizers can have a high degree of polarization with respect to near-infrared rays. In addition, since the linear polarizer is an absorption-type one that does not reflect near-infrared rays, the occurrence of reflected light that can serve as a noise can be suppressed.

The orientations of the transmission axes of the linear polarizer 120 and the linear polarizer 122 are not particularly limited as long as the transmission axis of the linear polarizer 120 and the transmission axis of the linear polarizer 122 are substantially orthogonal to each other. It is preferable that the transmission axis of the linear polarizer 120 of the first polarizing element 106a is set to have an orientation in which the transmitted linearly polarized light serves as p-polarized light with respect to a skin surface of the living body S. This makes it possible to suppress the reflection on a skin surface.

In addition, in the blood flow measurement device 100a, it is preferable that the near-infrared rays emitted from the light source unit 104 are configured to be tilted with respect to a skin surface of the living body S and to be incident in a direction in which the azimuth direction is toward the light receiving section 108. This makes it possible to increase the amount of received near-infrared rays scattered in the vicinity of the blood vessel in the light receiving section 108, and therefore, the SN ratio can be improved and the measurement accuracy can be improved.

A method for tilting the near-infrared rays emitted from the light source unit 104 with respect to a skin surface of the living body S is not particularly limited, and the light source unit 104 may be disposed in the control unit 102 (support substrate) such that the emission direction of the light source unit 104 is tilted with respect to the control unit 102 (main surface of the support substrate). Alternatively, the light source unit 104 may be configured to have a diffraction element or the like, or the first polarizing element 106a may be configured to have a diffraction element.

The linear polarizer formed of a liquid crystal compound will be described in detail later.

FIG. 3 is a conceptual view showing a part of another example of the blood flow measurement device of the embodiment of the present invention.

A blood flow measurement device 100b shown in FIG. 3 has a light source unit 104, a first polarizing element 106b, a light receiving section 108, and a second polarizing element 110b. Furthermore, in the blood flow measurement device 100b shown in FIG. 3, the control unit, the housing, and the like are not shown.

In the blood flow measurement device 100b shown in FIG. 3, the first polarizing element 106b has a linear polarizer 120 as the layer formed of the liquid crystal compound. Further, the first polarizing element 106b has a λ/4 plate 124 on the side of the linear polarizer 120 opposite to the side of the light source unit 104. In addition, in a preferred aspect, the second polarizing element 110b has a linear polarizer 122 as the layer formed of the liquid crystal compound. Further, the second polarizing element 110b has a λ/4 plate 125 on the side of the linear polarizer 122 opposite to the light receiving section 108. That is, the first polarizing element 106b and the second polarizing element 110b include a circularly polarizing plate composed of a linear polarizer and a λ/4 plate.

The λ/4 plate 124 of the first polarizing element 106b is disposed such that the linear polarizer 120 converts the near-infrared rays into circularly polarized light. That is, the λ/4 plate 124 is disposed such that the slow axis is at an angle of about 45° (or) −45° with respect to the transmission axis of the linear polarizer 120. Accordingly, the first polarizing element 106b changes the near-infrared rays emitted from the light source unit 104 into circularly polarized light.

The λ/4 plate 125 of the second polarizing element 110b converts circularly polarized light that is incident from the λ/4 plate 125 side into linearly polarized light. In addition, the λ/4 plate 125 is disposed such that the slow axis is at 45° (or) −45° with respect to the transmission axis of the linear polarizer 122. Such a second polarizing element 110b transmits one circularly polarized light of dextrorotatory circularly polarized light and levorotatory circularly polarized light, and cuts the other circularly polarized light. Specifically, the second polarizing element 110b transmits circularly polarized light having the same revolution direction as the circularly polarized light emitted from the first polarizing element 106b, and cuts circularly polarized light having an opposite revolution direction. Accordingly, for example, the second polarizing element 110b is disposed such that the orientation of the transmission axis of the linear polarizer 122 is the same as the orientation of the transmission axis of the linear polarizer 120 of the first polarizing element 106b and the orientation of the slow axis of the λ/4 plate 125 is the same as the orientation of the slow axis of the λ/4 plate 124 of the first polarizing element 106b. Alternatively, the second polarizing element 110b is disposed such that the orientation of the transmission axis of the linear polarizer 122 is orthogonal to the orientation of the transmission axis of the linear polarizer 120 of the first polarizing element 106b and the orientation of the slow axis of the λ/4 plate 125 is orthogonal to the orientation of the slow axis of the λ/4 plate 124 of the first polarizing element 106b. Hereinafter, a configuration in which the second polarizing element 110b is disposed such that the orientation of the transmission axis of the linear polarizer 122 is the same as the orientation of the transmission axis of the linear polarizer 120 of the first polarizing element 106b and the orientation of the slow axis of the λ/4 plate 125 is the same as the orientation of the slow axis of the λ/4 plate 124 of the first polarizing element 106b will be described as an example.

In such a blood flow measurement device 100b, in a case where near-infrared rays are irradiated from the light source unit 104, the linear polarizer 120 of the first polarizing element 106b changes the near-infrared rays into, for example, linearly polarized light that vibrates in the left-and-right direction in the drawing. The near-infrared rays that have been linearly polarized by the linear polarizer 120 are incident on the λ/4 plate 124 and are converted into circularly polarized light. For example, it is assumed that the near-infrared rays are converted into dextrorotatory circularly polarized light by the λ/4 plate 124. That is, the first polarizing element 106b converts the incident near-infrared rays into circularly polarized light. The near-infrared rays converted into dextrorotatory circularly polarized light are incident into the living body S. The near-infrared rays with which the living body S is irradiated are partially absorbed and scattered in the vicinity of the blood vessel. At this time, the near-infrared rays are depolarized from dextrorotatory circularly polarized light to serve as unpolarized light. A part of the scattered near-infrared rays travels toward the light receiving section 108 side and is incident on the second polarizing element 110b. The near-infrared rays are incident on the λ/4 plate 125 of the second polarizing element 110b, but are unpolarized light. Therefore, the near-infrared rays are incident on the linear polarizer 122 while remaining unpolarized. The linear polarizer 122 transmits the incident near-infrared rays as, for example, linearly polarized light that vibrates in the left-and-right direction in the drawing. The light receiving section 108 receives the linearly polarized near-infrared rays, converts the linearly polarized near-infrared rays into an electrical signal, and outputs the electrical signal to the control unit. The control unit performs various types of processing on the received data to calculate a blood flow change amount, a pulse rate, and the like.

On the other hand, a part of the near-infrared rays converted into dextrorotatory circularly polarized light by the first polarizing element 106b (linearly polarizer 120 and λ/4 plate 124) is reflected on a portion other than the measurement portion, such as a surface of a human body and an interface of an organ. At this time, the polarization is not eliminated, and the circularly polarized light is reflected such that the revolution direction is reversed. Therefore, the light is levorotatory circularly polarized light and is incident on the λ/4 plate 125 of the second polarizing element 110b. Since the slow axis of the λ/4 plate 125 is in the same direction as the slow axis of the λ/4 plate 124 of the first polarizing element 106b, the levorotatory circularly polarized light incident on the λ/4 plate 125 is converted into linearly polarized light that vibrates in the direction perpendicular to the paper plane in the drawing. This linearly polarized light is incident on the linear polarizer 122. Since the linear polarizer 122 has a transmission axis in the left-and-right direction in the drawing, it absorbs the linearly polarized light that vibrates in the direction perpendicular to the paper plane without transmitting the linearly polarized light. This makes it possible to cut the circularly polarized light reflected on a portion other than a measurement portion, that is, the near-infrared rays as the noise component, and makes it possible to suppress the light receiving section 108 from receiving the noise component.

Furthermore, even in a case where the second polarizing element 110b is configured to be disposed such that the orientation of the transmission axis of the linear polarizer 122 is orthogonal to the orientation of the transmission axis of the linear polarizer 120 of the first polarizing element 106b and the orientation of the slow axis of the λ/4 plate 125 is orthogonal to the orientation of the slow axis of the λ/4 plate 124 of the first polarizing element 106b, the circularly polarized light reflected on a portion other than a measurement portion can be cut.

Specifically, a part of the near-infrared rays changed into dextrorotatory circularly polarized light by the first polarizing element 106b is reflected on a portion other than the measurement portion, such as a surface of a human body and an interface of an organ, and thus, serves as levorotatory circularly polarized light and is incident on the λ/4 plate 125 of the second polarizing element 110b. Since the slow axis of the λ/4 plate 125 is orthogonal to the slow axis of the λ/4 plate 124 of the first polarizing element 106b, the levorotatory circularly polarized light incident on the λ/4 plate 125 is converted into linearly polarized light that vibrates in the left-and-right direction in the drawing. This linearly polarized light is incident on the linear polarizer 122. Since the linear polarizer 122 has a transmission axis in the direction perpendicular to the paper plane in the drawing, it absorbs the linearly polarized light that vibrates in the left-and-right direction without transmitting the linearly polarized light. This makes it possible to cut the circularly polarized light reflected on a portion other than a measurement portion, that is, the near-infrared rays as the noise component, and makes it possible to suppress the light receiving section 108 from receiving the noise component.

Here, the circularly polarized light has higher biological transmittance than the unpolarized light. Accordingly, a configuration in which circularly polarized light is incident into a living body can be adopted as the configuration in which a circularly polarizing plate is used as the first polarizing element 106b and the second polarizing element 110b as in the blood flow measurement device 100b can be adopted, and the amount of light scattered in the vicinity of the blood vessel can be increased, making it possible to further improve the SN ratio.

The λ/4 plate will be described in detail later.

In the examples shown in FIGS. 2 and 3, the first polarizing element and the second polarizing element have a function of changing the polarization state of near-infrared rays, but may further have a function of controlling a direction of near-infrared rays.

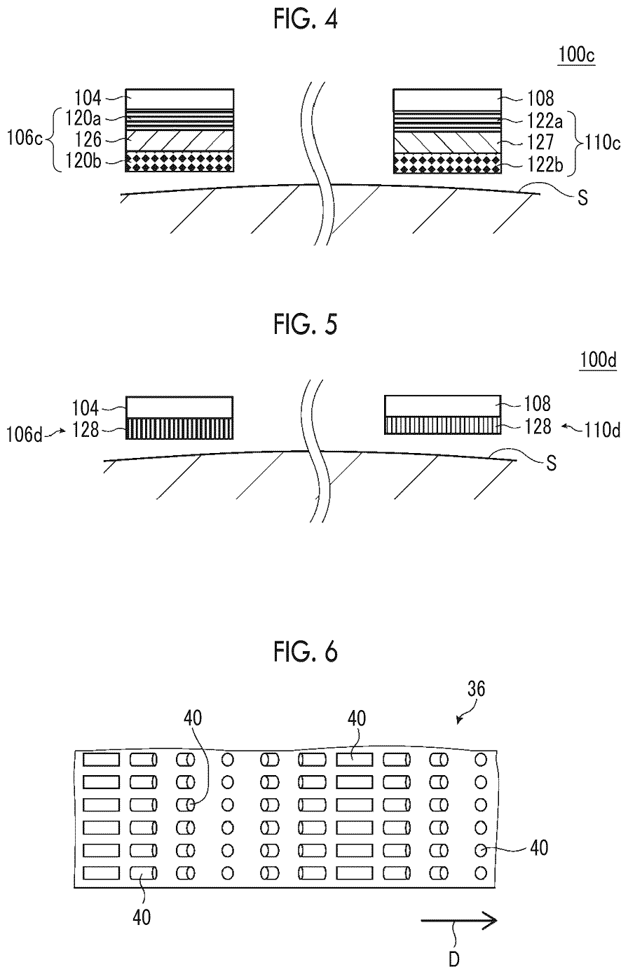

Δn example in which the first polarizing element and the second polarizing element further have a function of controlling a direction of near-infrared rays will be described with reference to FIGS. 4 and 5.

FIG. 4 is a conceptual view showing a part of another example of the blood flow measurement device of the embodiment of the present invention.

A blood flow measurement device 100c shown in FIG. 4 has a light source unit 104, a first polarizing element 106c, a light receiving section 108, and a second polarizing element 110c. Furthermore, in the blood flow measurement device 100c shown in FIG. 4, the control unit, the housing, and the like are not shown.

In the blood flow measurement device 100c shown in FIG. 4, the first polarizing element 106c has a first linear polarizer 120a, a retardation layer 126, and a second linear polarizer 120b in this order from the light source unit 104 side. The first linear polarizer 120a and the second linear polarizer 120b correspond to the layer formed of the liquid crystal compound in the present invention. In addition, as a preferred aspect, the second polarizing element 110c has a first linear polarizer 122a, a retardation layer 127, and a second linear polarizer 122b in this order from the light receiving section 108 side. The first linear polarizer 122a and the second linear polarizer 122b correspond to the layer formed of the liquid crystal compound in the present invention.

In the first polarizing element 106c, the first linear polarizer 120a and the second linear polarizer 120b are disposed such that transmission axes thereof are substantially orthogonal to each other. In the following description, it is assumed that the first linear polarizer 120a has a transmission axis in the left-and-right direction in the drawing and the second linear polarizer 120b has a transmission axis in the direction perpendicular to the paper plane.

The retardation layer 126 is configured to act as a λ/2 plate with respect to near-infrared rays having a wavelength emitted from the light source unit 104, which are incident from a direction tilted at a certain angle with respect to the main surface of the retardation layer 126. The retardation layer 126 is disposed such that the slow axis is at an angle of about 45° (or)−45° with respect to the transmission axis of the first linear polarizer 120a.

Similarly, in the second polarizing element 110c, the first linear polarizer 122a and the second linear polarizer 122b are disposed such that transmission axes thereof are substantially orthogonal to each other. In the following description, it is assumed that the first linear polarizer 122a has a transmission axis in the left-and-right direction in the drawing and the second linear polarizer 122b has a transmission axis in the direction perpendicular to the paper plane.

The retardation layer 127 is configured to act as a λ/2 plate with respect to near-infrared rays having a wavelength emitted from the light source unit 104, which are incident from a direction tilted at a certain angle with respect to the main surface of the retardation layer 127. The retardation layer 127 is disposed such that the slow axis is at an angle of about 45° (or)−45° with respect to the transmission axis of the first linear polarizer 122a.

In such a blood flow measurement device 100c, in a case where near-infrared rays are irradiated from the light source unit 104, the first linear polarizer 120a of the first polarizing element 106c changes the near-infrared rays into, for example, linearly polarized light that vibrates in the left-and-right direction in the drawing. The near-infrared rays that have been linearly polarized by the first linear polarizer 120a are incident on the retardation layer 126. The retardation layer 126 gives a phase difference to the incident linearly polarized near-infrared rays. Here, the linearly polarized light incident on the retardation layer 126 from a direction tilted by a certain angle α is given a phase difference of λ/2, and the vibration direction is rotated by 90°. That is, the linearly polarized light incident on the retardation layer 126 is changed to linearly polarized light that vibrates in the direction perpendicular to the paper plane in the drawing. On the other hand, the linearly polarized light incident in a direction tilted by an angle deviating from the angle α and the direction perpendicular to the main surface deviates from the phase difference of λ/2, and therefore, the rotation amount of the vibration direction deviates from 90°. The linearly polarized light of which the vibration direction is rotated by the retardation layer 126 is incident on the second linear polarizer 120b. Since the second linear polarizer 120b has a transmission axis in the direction perpendicular to the paper plane, the linearly polarized light incident from the direction tilted by the angle α transmits through the second linear polarizer 120b, and the linearly polarized light incident from a direction tilted by an angle deviating from the angle α and the direction perpendicular to the main surface is cut by the second linear polarizer 120b. Accordingly, the traveling direction of the near-infrared rays that have passed through the first polarizing element 106c is a direction tilted by the angle α.

As described above, the first polarizing element 106c can change the polarization state of the near-infrared rays emitted from the light source unit 104 and control the traveling direction of the near-infrared rays at the same time.

The near-infrared rays converted into linearly polarized light are incident into the living body S. The near-infrared rays with which the living body S is irradiated are partially absorbed and scattered in the vicinity of the blood vessel. At this time, the near-infrared rays are depolarized from linearly polarized light and thus serve as unpolarized light. A part of the scattered near-infrared rays travels toward the light receiving section 108 side and is incident on the second polarizing element 110c. The second linear polarizer 122b of the second polarizing element 110c converts the incident near-infrared rays into linearly polarized light that vibrates in the direction perpendicular to the paper plane. The near-infrared rays that have been linearly polarized by the second linear polarizer 122b are incident on the retardation layer 126. The retardation layer 126 gives a phase difference to the incident linearly polarized near-infrared rays. Here, the linearly polarized light incident on the retardation layer 126 from a direction tilted by a certain angle β is given a phase difference of λ/2 and the vibration direction is rotated by 90°. That is, the linearly polarized light incident on the retardation layer 126 is changed to linearly polarized light that vibrates in the left-and-right direction in the drawing. On the other hand, the linearly polarized light incident in a direction tilted by an angle deviating from the angle β and the direction perpendicular to the main surface deviates from the phase difference of λ/2, and therefore, the rotation amount of the vibration direction deviates from 90°. The linearly polarized light of which the vibration direction is rotated by the retardation layer 126 is incident on the first linear polarizer 122a. Since the first linear polarizer 122a has a transmission axis in the left-and-right direction in the drawing, the linearly polarized light incident from the direction tilted by the angle β transmits through the first linear polarizer 122a, and the linearly polarized light incident from a direction tilted by an angle deviating from the angle β and the direction perpendicular to the main surface is cut by the first linear polarizer 122a. Accordingly, the traveling direction of the near-infrared rays that have passed through the second polarizing element 110c is the direction tilted by the angle β. The near-infrared rays that have passed through the second polarizing element 110c are incident on the light receiving section 108. The light receiving section 108 receives the linearly polarized near-infrared rays, converts the linearly polarized near-infrared rays into an electrical signal, and outputs the electrical signal to the control unit. The control unit performs various types of processing on the received data to calculate a blood flow change amount, a pulse rate, and the like.

On the other hand, a part of the near-infrared rays that have been linearly polarized by the first polarizing element 106c is reflected on a portion other than the measurement portion, such as a surface of a human body and an interface of an organ. At this time, since the polarization is not eliminated, the linearly polarized light that vibrates in the direction perpendicular to the paper plane in the drawing is incident on the second linear polarizer 122b of the second polarizing element 110c. Since the second linear polarizer 122b of the second polarizing element 110c has a transmission axis in the direction perpendicular to the paper plane, it transmits the linearly polarized light that vibrates in the direction perpendicular to the paper plane. The near-infrared rays that have been linearly polarized by the second linear polarizer 122b are incident on the retardation layer 126. The retardation layer 126 gives a phase difference to the incident linearly polarized near-infrared rays. Here, the linearly polarized light incident on the retardation layer 126 from the direction tilted by the angle β is given a phase difference of λ/2 and the vibration direction is rotated by 90°. However, the light reflected on a portion other than the measurement portion is incident from a direction tilted by an angle deviating from the angle β. Therefore, the phase difference by the retardation layer deviates from λ/2 and the rotation amount of the vibration direction deviates from 90°. The linearly polarized light of which the vibration direction is rotated by the retardation layer 126 is incident on the first linear polarizer 122a. Since the first linear polarizer 122a has a transmission axis in the left-and-right direction, the linearly polarized light incident from a direction tilted by an angle deviating from the angle β is cut by the first linear polarizer 122a.

This makes it possible to cut the linearly polarized light reflected on a portion other than the measurement portion, that is, the near-infrared rays as a noise component, and makes it possible to suppress the light receiving section 108 from receiving the noise component.

By allowing the first polarizing element 106c to control the traveling direction of the near-infrared rays to a direction in which the azimuth direction is toward the light receiving section 108 (second polarizing element 110c) and a direction tilted at a predetermined angle with respect to the perpendicular line of the main surface of the first polarizing element 106c, it is possible to increase the amount of light toward the light receiving section 108 (second polarizing element 110c) among the near-infrared rays that are irradiated into the living body S and scattered in the vicinity of the blood vessel. Thus, the SN ratio can be further improved.

Furthermore, in the example shown in FIG. 4, the first polarizing element 106c and the second polarizing element 110c are configured to have the first linear polarizer, the retardation layer, and the second linear polarizer in this order, but the present invention is not limited thereto. Either of the first polarizing element 106c and the second polarizing element 110c may be configured to have the first linear polarizer, the retardation layer, and the second linear polarizer in this order. In this case, the other polarizer may be configured to consist of, for example, a linear polarizer, and the linearly polarized light reflected on a portion other than the measurement portion may be cut by the second polarizing element.

In addition, in the example shown in FIG. 4, the first linear polarizer and the second linear polarizer in the first polarizing element 106c and the second polarizing element 110c are configured to be disposed such that the transmission axes are orthogonal to each other, but may be configured to be disposed such that the transmission axes are parallel to each other. In a case where the transmission axes of the first linear polarizer and the second linear polarizer are parallel to each other, for example, in a case where the direction such that the refractive index of the retardation layer is 0, that is, the direction of the optical axis of the retardation layer is tilted in a range of 20 degrees to 60 degrees with respect to the main surface, the amount of light toward the light receiving section 108 (second polarizing element 110c) can be increased. Thus, the SN ratio can be further improved, which is preferable.

In addition, in the example shown in FIG. 4, in the first polarizing element 106c and/or the second polarizing element 110c, the second linear polarizer may be configured to have an absorption axis in the direction perpendicular to the surface. In this case, the absorption axes of the first linear polarizer and the second linear polarizer can be set to be in a relationship of being orthogonal or parallel to each other only with respect to the near-infrared rays incident from the oblique direction. This makes it possible to reduce the range of the angle at which the obliquely reflected light from the measurement portion is transmitted, and as a result, the polarizer and the retardation layer can be set to have an axial angle relationship in which the transmission rays are reduced immediately after the angle is slightly changed from the most transmitted angle. As a result, it is possible to perform measurement with more weight placed on the reflected light at a required angle, and it is thus possible to perform measurement with less noise.

FIG. 5 is a conceptual view showing a part of another example of the blood flow measurement device of the embodiment of the present invention.

A blood flow measurement device 100d shown in FIG. 5 has a light source unit 104, a first polarizing element 106d, a light receiving section 108, and a second polarizing element 110d. Furthermore, in the blood flow measurement device 100d shown in FIG. 5, the control unit, the housing, and the like are not shown.

In the blood flow measurement device 100d shown in FIG. 5, the first polarizing element 106d has, as the layer formed of the liquid crystal compound, an optically anisotropic layer having a liquid crystal alignment pattern in which an orientation of an optical axis derived from the liquid crystal compound changes while continuously rotating along at least one in-plane direction. In addition, as a preferred aspect, the second polarizing element 110d has, as the layer formed of the liquid crystal compound, an optically anisotropic layer having a liquid crystal alignment pattern in which an orientation of an optical axis derived from the liquid crystal compound changes while continuously rotating along at least one in-plane direction.

The optically anisotropic layer having a liquid crystal alignment pattern in which an orientation of an optical axis derived from a liquid crystal compound changes while continuously rotating along at least one in-plane direction is a liquid crystal diffraction element that diffracts incident near-infrared rays. In addition, the liquid crystal diffraction element diffracts a dextrorotatory circularly polarized light component and a levorotatory circularly polarized light component of incident near-infrared rays in different directions.

The liquid crystal diffraction element will be described in detail later.

In such a blood flow measurement device 100d, in a case where near-infrared rays are irradiated from the light source unit 104, the liquid crystal diffraction element 128 of the first polarizing element 106d diffracts the near-infrared rays, for example, a dextrorotatory circularly polarized light component in a direction in which the azimuth direction is toward the light receiving section 108 (second polarizing element 110d) at a predetermined angle tilted with respect to the perpendicular line of the main surface of the liquid crystal diffraction element 128. The near-infrared rays that are dextrorotatory circularly polarized by the liquid crystal diffraction element 128 (first polarizing element 106d) are incident into the living body S. The near-infrared rays with which the living body S is irradiated are partially absorbed and scattered in the vicinity of the blood vessel. At this time, the near-infrared rays are depolarized from dextrorotatory circularly polarized light to serve as unpolarized light. A part of the scattered near-infrared rays travels toward the light receiving section 108 side and is incident on the second polarizing element 110d. The liquid crystal diffraction element 128 of the second polarizing element 110d diffracts a dextrorotatory circularly polarized light component or a levorotatory circularly polarized light component of unpolarized near-infrared rays incident from an oblique direction in a direction toward the light receiving section 108 to transmit the light. The light receiving section 108 receives the circularly polarized near-infrared rays, converts the light into an electrical signal, and thus, outputs the electrical signal to the control unit. The control unit performs various types of processing on the received data to calculate a blood flow change amount, a pulse rate, and the like.

On the other hand, a part of the near-infrared rays that are dextrorotatory circularly polarized by the first polarizing element 106d is reflected on a portion other than the measurement portion, such as a surface of a human body and an interface of an organ. At this time, the polarization is not eliminated, and the circularly polarized light is reflected, reversing the revolution direction, and therefore, the circularly polarized light is levorotatory circularly polarized light and is incident on the liquid crystal diffraction element 128 of the second polarizing element 110d. The liquid crystal diffraction element 128 does not diffract the incident levorotatory circularly polarized light in the direction of the light receiving section 108, and diffracts the dextrorotatory circularly polarized light generated as a result of depolarization in the measurement portion in the direction of the light receiving section 108. This makes it possible to cut the circularly polarized light reflected on a portion other than a measurement portion, that is, the near-infrared rays as the noise component, and makes it possible to suppress the light receiving section 108 from receiving the noise component.

Furthermore, in the example shown in FIG. 5, the first polarizing element 106d and the second polarizing element 110d are configured to have the liquid crystal diffraction element, but the present invention is not limited thereto, and any one of the first polarizing element 106d or the second polarizing element 110d may be configured to have the liquid crystal diffraction element. For example, in a case where the first polarizing element is a liquid crystal diffraction element, the second polarizing element may be configured to have, for example, a circularly polarizing plate (a linear polarizer+a λ/4 plate) and may be configured to cut circularly polarized light reflected on a portion other than the measurement portion.

### <Linear Polarizer>

The linear polarizers 120, 120a, 120b, 122, 122a, and 122b are layers formed of a liquid crystal compound. These are absorption-type polarizers, which absorb linearly polarized light that vibrates in the absorption axis direction in the incidence rays, and transmit linearly polarized light that vibrates in the transmission axis direction.

The liquid crystal compound may be a rod-like liquid crystal compound or a disk-like liquid crystal compound.

The liquid crystal compound may have a polymerizable group. Examples of the liquid crystal compound (polymerizable liquid crystal compound) having a polymerizable group include compounds exemplified by the polymerizable liquid crystal compound which will be described later in the optically anisotropic layer.

The liquid crystal compound may be a thermotropic liquid crystal compound or a lyotropic liquid crystal compound. Furthermore, the lyotropic liquid crystal compound is a liquid crystal compound that exhibits a property of causing a phase transition between an isotropic phase and a liquid crystal phase by changing a temperature or a concentration in a solution state in which the liquid crystal compound is dissolved in a solvent.

Examples of the lyotropic liquid crystal compound include the non-colorable lyotropic liquid crystal compounds (for example, a rod-like compound and a plate-like compound) described in paragraphs to of WO2021/200987A.

It is preferable that the linear polarizer is formed of a liquid crystal composition including a liquid crystal compound and a dichroic substance.

The liquid crystal compound included in the liquid crystal composition is as described above.

The dichroic substance refers to a compound having a property in which an absorbance in a major axis direction of a molecule and an absorbance in a minor axis direction of the molecule are different from each other.

The dichroic substance preferably has a maximum absorption wavelength in a near-infrared region. More specifically, the maximum absorption wavelength of the dichroic substance is preferably in a wavelength range of 700 to 1,600 nm, more preferably in a wavelength range of 700 to 1,200 nm, and still more preferably in a wavelength range of 700 to 900 nm.

That is, the dichroic substance is preferably a so-called near-infrared absorbing dye.

The dichroic substance may or may not exhibit liquid crystallinity (for example, lyotropic liquid crystallinity).

The type of the dichroic substance is not particularly limited, but a cyanine-based coloring agent, an oxonol-based coloring agent, a boron complex-based coloring agent, a phthalocyanine-based coloring agent, a squarylium-based coloring agent, a metal complex-based coloring agent, a diimmonium-based coloring agent, or a perylene-based coloring agent is preferable.

In a case where a linear polarizer is produced using the liquid crystal composition, examples of the production method include a method in which the liquid crystal composition is applied to form a coating film, and optionally, the formed coating film is subjected to an alignment treatment to produce a linear polarizer.

A method for applying the liquid crystal composition is not particularly limited, and examples thereof include known methods such as spin coating and bar coating.

The substrate on which the liquid crystal composition is applied may have an alignment film on a surface thereof. By providing the alignment film, the liquid crystal compound is aligned according to the alignment restriction force of the alignment film.

The formed coating film is subjected to an alignment treatment as necessary. Examples of the alignment treatment include an optimum method depending on the type of the liquid crystal compound used.

For example, in a case where the liquid crystal compound is a thermotropic liquid crystal compound and the above-described alignment film is used, the liquid crystal compound can be aligned by subjecting the coating film to a heat treatment.

In addition, in a case where the liquid crystal compound is a lyotropic liquid crystal compound, it is possible to simultaneously perform two treatments of coating and aligning the compound by adopting a coating method of applying shearing to the liquid crystal composition, such as wire bar coating.

The formed coating film may be subjected to a hardening treatment as necessary. In particular, in a case where the liquid crystal compound has a polymerizable group, the polymerizable groups can be polymerized by performing a heating treatment or a light irradiation treatment.

By carrying out the procedure, the dichroic substance is also aligned along the alignment of the liquid crystal compound and a linear polarizer having predetermined characteristics is obtained.

### <λ/4 Plate>

The λ/4 plates 124 and 125 function as λ/4 plates with respect to the wavelength of incidence rays, and can convert linearly polarized light into circularly polarized light and convert circularly polarized light into linearly polarized light. The λ/4 plate is not particularly limited as long as it can convert incident linearly polarized light into circularly polarized light and convert incident circularly polarized light into linearly polarized light, and well-known λ/4 plates in the related art can be used.

In the present invention, from the viewpoint of wide angle characteristics and wide wavelength dispersibility, the λ/4 plate is preferably a layer formed of a liquid crystal compound.

The wide angle characteristics are a range of angles (angles of incidence rays with respect to perpendicular line of the main surface of the λ/4 plate) at which a phase difference of λ/4 can be imparted in a case where near-infrared rays are incident from an oblique direction with respect to the λ/4 plate, that is, a range of angles at which the plate functions as a λ/4 plate.

From the viewpoint that the plate can function as a λ/4 plate in a wider angle range, the λ/4 plate is preferably a laminate of a layer formed of a rod-like liquid crystal compound (for example, a layer formed by fixing a rod-like compound horizontally aligned) and a layer formed of a disk-like liquid crystal compound (for example, a layer formed by fixing a disk-like liquid crystal compound vertically aligned). Examples of each layer constituting such a laminate include the layers described in JP6975074B and JP6640847B.

Alternatively, the λ/4 plate is preferably a laminate of a layer in which a rod-like liquid crystal compound is horizontally aligned (for example, a layer formed by fixing a horizontally aligned rod-like compound) and a layer in which a rod-like liquid crystal compound is vertically aligned (for example, a layer formed by fixing a vertically aligned rod-like compound). Examples of each layer constituting such a laminate include the layers described in WO2019/159960A.

The wavelength dispersibility is a wavelength range exhibiting ¼ wavelength characteristics. As described above, in the blood flow measurement device of the embodiment of the present invention, a configuration in which two or more kinds of near-infrared rays having different wavelengths are used is preferably used. In this case, it is preferable that the λ/4 plate exhibits ¼ wavelength characteristics at any wavelength, and it is also preferable that the λ/4 plate exhibits so-called reverse wavelength dispersibility (a property in which an in-plane retardation increases as a measurement wavelength increases).

From the viewpoint of the wavelength dispersibility, it is preferable that the λ/4 plate is a layer formed of a reverse dispersion liquid crystal compound. Examples of the layer formed of the reverse dispersion liquid crystal compound include the layers described in WO2019/159960A.

In addition, the λ/4 plate may be a laminate of a λ/4 plate and a λ/2 plate. Examples of each layer constituting such a laminate include the layers described in JP6975074B and JP6640847B.

In addition, the λ/4 plate may include a layer formed by fixing a liquid crystal compound twistedly aligned along a helical axis extending along a thickness direction. Examples of the form including the layer formed by fixing a liquid crystal compound twistedly aligned along a helical axis extending along the thickness direction include the layer described in WO2021/033631A.

As described above, the λ/4 plate may be formed of a liquid crystal compound.

The liquid crystal compound may be a rod-like liquid crystal compound or a disk-like liquid crystal compound.

The liquid crystal compound may have a polymerizable group. Examples of the liquid crystal compound (polymerizable liquid crystal compound) having a polymerizable group include compounds exemplified by the polymerizable liquid crystal compound which will be described later in the optically anisotropic layer.

In addition, as described above, the liquid crystal compound may be a liquid crystal compound having forward wavelength dispersibility or a liquid crystal compound having reverse wavelength dispersibility.

A method for producing the λ/4 plate formed of the liquid crystal compound is not particularly limited, and a known method can be adopted. Examples thereof include a method in which a liquid crystal composition including a liquid crystal compound is applied onto a substrate having an alignment film, and the coating film is subjected to an alignment treatment (for example, a heating treatment), and as necessary, further to a hardening treatment.

### <Retardation Layer>

In the retardation layer, a phase difference (optical path difference) is provided for two orthogonal polarized light components to change the state of the incident polarized light. In the present invention, the retardation layer is a layer in which materials having birefringence, such as liquid crystal compounds, are arranged in the same direction.

As described in FIG. 4, it is preferable that the retardation layer used in the polarizing element controlling the direction of near-infrared rays functions as a λ/2 plate with respect to near-infrared rays incident from a direction tilted at a certain angle from the viewpoint of transmitting near-infrared rays in a direction tilted at a certain angle. From this point, it is preferable that the retardation layer is one in which the liquid crystal compounds are obliquely aligned with respect to the main surface.

As described above, in the blood flow measurement device of the embodiment of the present invention, a configuration in which two or more kinds of near-infrared rays having different wavelengths are used is preferably used. In this case, the retardation layer preferably exhibits a predetermined phase difference at any wavelength, and more preferably exhibits so-called reverse wavelength dispersibility.

The retardation layer may be formed of a liquid crystal compound.

The liquid crystal compound may be a rod-like liquid crystal compound or a disk-like liquid crystal compound.

The liquid crystal compound may have a polymerizable group. Examples of the liquid crystal compound (polymerizable liquid crystal compound) having a polymerizable group include compounds exemplified by the polymerizable liquid crystal compound which will be described later in the optically anisotropic layer.

A method for producing the retardation layer formed of the liquid crystal compound is not particularly limited, and a known method can be adopted. Examples thereof include a method in which a liquid crystal composition including a liquid crystal compound is applied onto a substrate having an alignment film, and the coating film is subjected to an alignment treatment (for example, a heating treatment), and as necessary, further to a hardening treatment.

### <Liquid Crystal Diffraction Element>

The liquid crystal diffraction element has an optically anisotropic layer in which liquid crystal compounds are aligned in a predetermined arrangement, and bends near-infrared rays by diffraction.

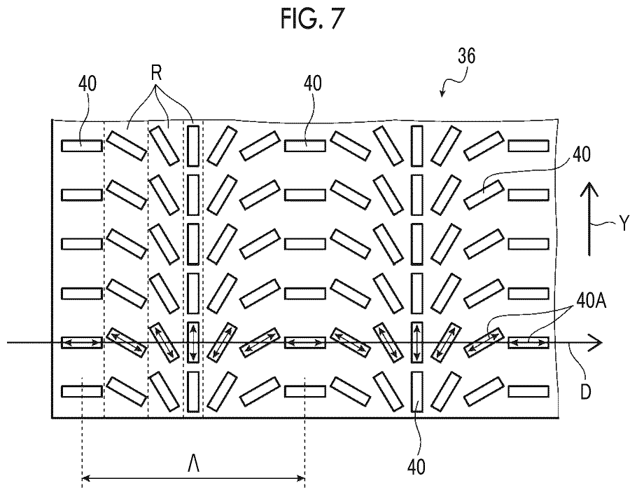

The optically anisotropic layer contained in the liquid crystal diffraction element will be described with reference to FIGS. 6 and 7.

The optically anisotropic layer shown in FIGS. 6 and 7 is layer formed by fixing a liquid crystal phase where a liquid crystal compound is aligned and has a liquid crystal alignment pattern in which an orientation of an optical axis derived from the liquid crystal compound changes while continuously rotating along at least one in-plane direction.

In the optically anisotropic layer, as conceptually shown in FIG. 6, a liquid crystal compound 40 is not helically twisted and rotated in a thickness direction, and the liquid crystal compounds 40 at the same position in the plane direction are aligned such that the orientations of optical axes 40A thereof are the same orientations.

### <<Liquid Crystal Alignment Pattern of Optically Anisotropic Layer>>

The optically anisotropic layer has the liquid crystal alignment pattern in which the orientation of the optical axis 40A derived from the liquid crystal compound 40 changes while continuously rotating in one direction in the plane of the optically anisotropic layer.

Furthermore, the optical axis 40A derived from the liquid crystal compound 40 is an axis having the highest refractive index in the liquid crystal compound 40, that is, a so-called slow axis. For example, in a case where the liquid crystal compound 40 is a rod-like liquid crystal compound, the optical axis 40A is directed along a rod-like major axis direction. In the following description, the optical axis 40A derived from the liquid crystal compound 40 will also be referred to as “the optical axis 40A of the liquid crystal compound 40” or “the optical axis 40A”.

FIG. 7 conceptually shows a plan view of the optically anisotropic layer.

Furthermore, the plan view is a view in which the optically anisotropic layer is seen from the top in FIG. 6, that is, a view in which the optically anisotropic layer is seen from the thickness direction (the laminating direction of the respective layers (films)).

In addition, in FIG. 7, in order to clarify the configuration of the optically anisotropic layer, only the liquid crystal compound 40 on the surface is shown as the liquid crystal compound 40.

As shown in FIG. 7, on the surface, the liquid crystal compound 40 forming the optically anisotropic layer has the liquid crystal alignment pattern in which the orientation of the optical axis 40A changes while continuously rotating along a predetermined one direction indicated by an arrow D (hereinafter referred to as the arrangement axis D) in the plane of the optically anisotropic layer. In the example shown in the drawing, the liquid crystal compound 40 has the liquid crystal alignment pattern in which the optical axis 40A of the liquid crystal compound 40 changes while continuously rotating clockwise along the arrangement axis D direction.

The liquid crystal compounds 40 forming the optically anisotropic layer are in a state of being two-dimensionally arranged along the arrangement axis D and in a direction orthogonal to one direction of the arrangement axis D (arrangement axis D direction).

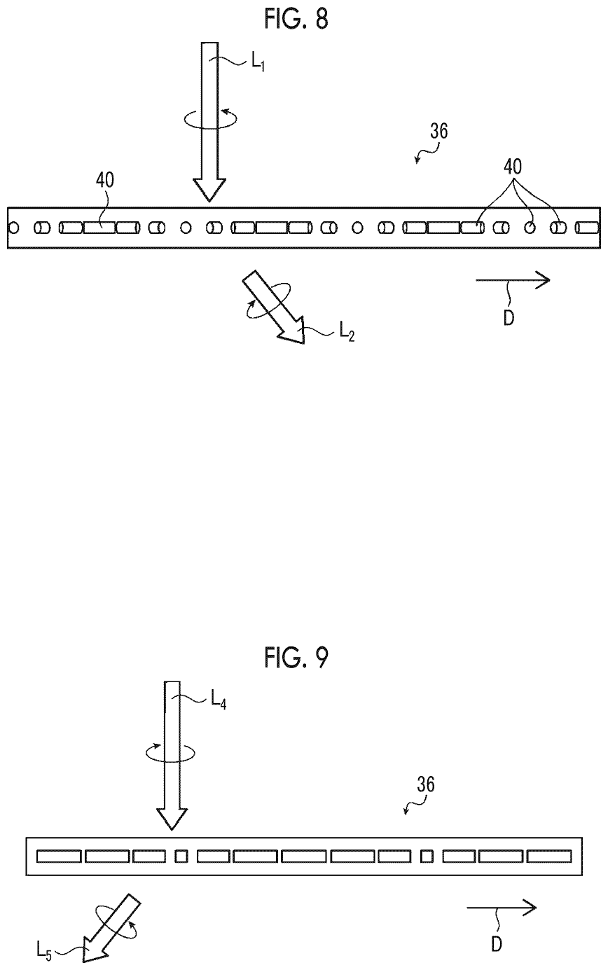

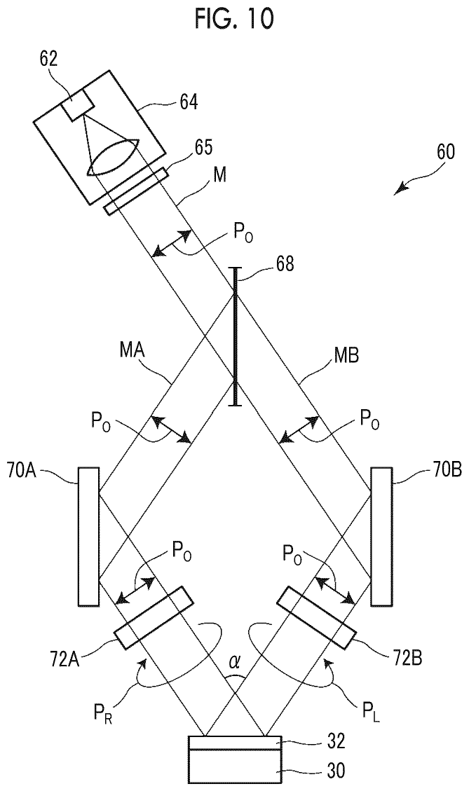

In the following description, a direction orthogonal to the arrangement axis D direction will be referred to as a “Y direction” for convenience of description. That is, the arrow Y direction is a direction orthogonal to the one direction in which the orientation of the optical axis 40A of the liquid crystal compound 40 changes while continuously rotating in the optically anisotropic layer. Accordingly, in FIGS. 8 and 9 which will be described later, the Y direction is the direction orthogonal to the paper plane.

Specifically, the expression that the orientation of the optical axis 40A of the liquid crystal compound 40 changes while continuously rotating in the arrangement axis D direction (one predetermined direction) means that an angle formed between the optical axis 40A of the liquid crystal compound 40, which is arranged along the arrangement axis D direction, and the arrangement axis D direction varies depending on positions in the arrangement axis D direction, and the angle formed between the optical axis 40A and the arrangement axis D direction sequentially changes from θ to θ+180° or θ-180° along the arrangement axis D direction.

Furthermore, a difference between the angles of the optical axes 40A of the liquid crystal compounds 40 adjacent to each other in the arrangement axis D direction is preferably 45° or less, more preferably 15° or less, and still more preferably the less angles.

In addition, in the present invention, with regard to a rotation direction of the optical axis 40A of the liquid crystal compound in the arrangement axis D direction, the liquid crystal compounds 40 (the optical axes 40A) rotate in an orientation in which the angle formed between the optical axes 40A of the liquid crystal compounds 40 adjacent to each other in the arrangement axis D direction decreases. Accordingly, in the optically anisotropic layer shown in FIG. 6 and FIG. 7, the optical axis 40A of the liquid crystal compound 40 rotates to the right (clockwise) along the direction indicated by the arrow of the arrangement axis D.

On the other hand, in the liquid crystal compound 40 forming the optically anisotropic layer, the orientations of the optical axes 40A are the same in the Y direction orthogonal to the arrangement axis D direction, that is, the Y direction orthogonal to one direction in which the optical axis 40A continuously rotates.

In other words, in the liquid crystal compound 40 forming the optically anisotropic layer, angles formed between the optical axes 40A of the liquid crystal compound 40 and the arrangement axis D direction are the same in the Y direction.

In the liquid crystal compounds arranged in the Y direction in the optically anisotropic layer, the angles formed between the optical axes 40A and the arrangement axis D direction (the one direction in which the orientation of the optical axis of the liquid crystal compound 40 rotates) are the same. A region in which the liquid crystal compounds 40 in which the angles formed between the optical axes 40A and the arrangement axis D direction are the same are arranged in the Y direction will be referred to as a region R.

In this case, it is preferable that the value of the in-plane retardation (Re) in each region R is a half wavelength, that is, λ/2. The in-plane retardation is calculated from the product of a difference Δn in refractive index generated by refractivity anisotropy of the region R and the thickness of the optically anisotropic layer. Here, the difference in refractive index generated by refractive index anisotropy of the region R in the optically anisotropic layer is defined by a difference between a refractive index of a direction of an in-plane slow axis of the region R and a refractive index of a direction orthogonal to the direction of the slow axis. That is, the difference in refractive index Δn due to the refractive index anisotropy of the regions R is the same as a difference between a refractive index of the liquid crystal compound 40 in the direction of the optical axis 40A and a refractive index of the liquid crystal compound 40 in the direction perpendicular to the optical axis 40A in the plane of the region R. That is, the difference Δn in refractive index is the same as the difference in refractive index of the liquid crystal compound 40.

In the optically anisotropic layer, in the liquid crystal alignment pattern of such a liquid crystal compound 40, a length (distance) over which the optical axis 40A of the liquid crystal compound 40 rotates by 180° in the arrangement axis D direction in which the optical axis 40A changes while continuously rotating in the plane is a length A of a single period in the liquid crystal alignment pattern.

That is, a distance between the centers of two liquid crystal compounds 40 in the arrangement axis D direction is the length A of the single period, the two liquid crystal compounds 40 having the same angle in the arrangement axis D direction. Specifically, as shown in FIG. 7, a distance between centers in the arrangement axis D direction of two liquid crystal compounds 40 in which the arrangement axis D direction and the direction of the optical axis 40A match each other is the length A of the single period. In the following description, the length Λ of the single period will also be referred to as a “single period Λ”.

In the liquid crystal alignment pattern of the optically anisotropic layer, the single period Λ is repeated in the arrangement axis D direction, that is, in the one direction in which the orientation of the optical axis 40A changes while continuously rotating.

In a case where circularly polarized light is incident on such an optically anisotropic layer, the light is refracted such that the direction of the circularly polarized light is converted.

This action is conceptually shown in FIGS. 8 and 9. Furthermore, in the optically anisotropic layer, the value of the product of the difference in refractive index of the liquid crystal compound and the thickness of the optically anisotropic layer is λ/2.

As shown in FIG. 8, in a case where a value of the product of a difference in refractive index of the liquid crystal compound of the optically anisotropic layer and a thickness of the optically anisotropic layer is λ/2, and incidence rays L₁ that are levorotatory circularly polarized light are incident on the optically anisotropic layer, the incidence rays L₁ pass through the optically anisotropic layer to be imparted with a phase difference of 180° such that transmission rays L₂ are converted into dextrorotatory circularly polarized light.

In addition, the liquid crystal alignment pattern formed in the optically anisotropic layer is a pattern that is periodic in the arrangement axis D direction. Therefore, the transmission rays L₂ travel in a direction different from a traveling direction of the incidence rays L₁. In this way, the levorotatory circularly polarized incidence rays L₁ are converted into dextrorotatory circularly polarized transmission rays L₂, which are tilted by a predetermined angle in the arrangement axis D direction with respect to an incidence direction. In the example shown in FIG. 8, the transmission rays L₂ are diffracted to travel in the lower right direction.

On the other hand, in a case where the value of the product of a difference in refractive index of the liquid crystal compound of the optically anisotropic layer and a thickness of the optically anisotropic layer is λ/2, as shown in FIG. 9, as incidence rays L₄ of dextrorotatory circularly polarized light is incident on the optically anisotropic layer, the incidence rays L₄ pass through the optically anisotropic layer, thereby imparting a phase difference of 180°, and are converted into levorotatory circularly polarized transmission rays L₅.

In addition, since the liquid crystal alignment pattern formed in the optically anisotropic layer is a pattern that is periodic in the arrangement axis D direction, the transmission rays L₅ travel in a direction different from a traveling direction of the incidence rays L₄. In this case, the transmission rays L₅ travel in a direction different from the transmission rays L₂, that is, in a direction opposite to the arrow direction of the arrangement axis D with respect to the incidence direction. In this way, the incidence rays L₄ are converted into levorotatory circularly polarized transmission rays L₅, which are tilted by a predetermined angle in a direction opposite to the arrangement axis D direction with respect to the incidence direction. In the example shown in FIG. 9, the transmission rays L₅ are diffracted to travel in the lower left direction.

As described above, refraction angles of the transmission ray components L₂ and L₅ can be adjusted depending on the length of the single period Λ of the liquid crystal alignment pattern formed in the optically anisotropic layer. Specifically, also in the optically anisotropic layer, as the single period Λ of the liquid crystal alignment pattern decreases, light components transmitted through the liquid crystal compounds 40 adjacent to each other more strongly interfere with each other. Therefore, the transmission ray components L₂ and L₅ can be more largely refracted.

In addition, by reversing the rotation direction of the optical axis 40A of the liquid crystal compound 40 that rotates along the arrangement axis D direction, the azimuth direction of the refraction of transmission rays can be reversed. That is, in the example FIGS. 8 and 9, the rotation direction of the optical axis 40A toward the arrangement axis D direction is clockwise. By setting this rotation direction to be counterclockwise, the azimuth direction of the refraction of transmission rays can be reversed. Specifically, in FIGS. 8 and 9, in a case where the rotation direction of the optical axis 40A toward the arrangement axis D direction is counterclockwise, levorotatory circularly polarized light incident on the optically anisotropic layer from the upper side in the drawing is transmitted through the optically anisotropic layer such that the transmission rays are converted into dextrorotatory circularly polarized light and is diffracted to travel in the lower left direction in the drawing. In addition, dextrorotatory circularly polarized light incident on the optically anisotropic layer from the upper side in the drawing is transmitted through the optically anisotropic layer such that the transmission rays are converted into levorotatory circularly polarized light and is diffracted to travel in the lower right direction in the drawing.

### <<Method for Forming Optically Anisotropic Layer>>

For example, a method for forming the optically anisotropic layer has a step of applying a liquid crystal composition including the prepared liquid crystal compound to the alignment film; and a step of hardening the applied liquid crystal composition.

The preparation of the liquid crystal composition may be performed using a well-known method in the related art. Furthermore, for the application of the liquid crystal composition, various known methods used for the application of a liquid, such as printing methods such as ink jet and scroll printing, spin coating, bar coating, gravure coating, and spray coating can be used. In addition, as the coating thickness of the liquid crystal composition (the thickness of the coating film), a coating thickness that can be obtained an optically anisotropic layer having a desired thickness may be appropriately set depending on the liquid crystal composition and the like.

Here, the alignment pattern is formed on the alignment film as described later, and therefore, the liquid crystal compound of the liquid crystal composition applied to the alignment film is aligned along the alignment pattern (anisotropic period pattern) of the alignment film.

The liquid crystal composition is optionally dried and/or heated and then cured. The liquid crystal composition may be cured using a well-known method such as photopolymerization or thermal polymerization. For the polymerization, photopolymerization is preferable. With regard to the light irradiation, ultraviolet ray is preferably used. The irradiation energy is preferably 20 mJ/cm² to 50 J/cm², and more preferably 50 to 1,500 mJ/cm². In order to promote a photopolymerization reaction, the light irradiation may be performed under heating conditions or in a nitrogen atmosphere. A wavelength of the ultraviolet rays to be emitted is preferably 250 to 430 nm. In the case of heating, the heating temperature is preferably 200° C. or lower, and more preferably 130° C. or lower.

By hardening the liquid crystal composition, the liquid crystal compound in the liquid crystal composition is fixed in a state (liquid crystal alignment pattern) where the liquid crystal compound is aligned along the alignment pattern of the alignment film. As a result, an optically anisotropic layer having a liquid crystal alignment pattern in which an orientation of an optical axis derived from the liquid crystal compound changes while continuously rotating along at least one in-plane direction is formed.

Furthermore, in a case where the optically anisotropic layer is completed, the liquid crystal compound does not have to exhibit liquid crystallinity. For example, the polymerizable liquid crystal compound may be made to have a high molecular weight by a hardening reaction and therefore the liquid crystallinity may be lost.

In addition, the optically anisotropic layer may be formed by applying multiple layers of the liquid crystal composition to the alignment film. The multilayer application refers to a method for forming the optically anisotropic layer by repeating the following processes until a desired thickness is obtained, the processes including: forming a first liquid crystal fixed layer by applying the liquid crystal composition for forming the first layer to the alignment film, heating the liquid crystal composition, cooling the liquid crystal composition, and irradiating the liquid crystal composition with ultraviolet light for hardening; and forming a second or subsequent liquid crystal fixed layer by applying the liquid crystal composition for forming the second or subsequent layer to the formed liquid crystal fixed layer, heating the liquid crystal composition, cooling the liquid crystal composition, and irradiating the liquid crystal composition with ultraviolet light for hardening as described above. By forming the liquid crystal layer by the multilayer coating, the total thickness of the liquid crystal layer can be increased. In addition, even in a case where the total thickness of the liquid crystal layer is increased, the alignment direction of the alignment film is projected from a lower surface to an upper surface of the liquid crystal layer.

### <Liquid Crystal Composition for Forming Optically Anisotropic Layer>

Examples of a material used for forming the optically anisotropic layer include a liquid crystal composition including a liquid crystal compound. It is preferable that the liquid crystal compound is a polymerizable liquid crystal compound.

In addition, the liquid crystal composition used for forming the liquid crystal layer may further include a surfactant and a chiral agent.

### —Polymerizable Liquid Crystal Compound—

The polymerizable liquid crystal compound may be a rod-like liquid crystal compound or a disk-like liquid crystal compound.

Examples of the rod-like polymerizable liquid crystal compound include a rod-like nematic liquid crystal compound. As the rod-like nematic liquid crystal compound, an azomethine compound, an azoxy compound, a cyanobiphenyl compound, a cyanophenyl ester compound, a benzoate compound, a phenyl cyclohexanecarboxylate compound, a cyanophenylcyclohexane compound, a cyano-substituted phenylpyrimidine compound, an alkoxy-substituted phenylpyrimidine compound, a phenyldioxane compound, a tolane compound, or an alkenylcyclohexylbenzonitrile compound is preferably used. Not only a low-molecular-weight liquid crystal compound but also a high-molecular-weight liquid crystal compound can be used.

The polymerizable liquid crystal compound can be obtained by introducing a polymerizable group into the liquid crystal compound. Examples of the polymerizable group include an unsaturated polymerizable group, an epoxy group, and an aziridinyl group, and among these, the unsaturated polymerizable group is preferable, and the ethylenically unsaturated polymerizable group is more preferable. The polymerizable group can be introduced into a molecule of the liquid crystal compound by various methods. The number of the polymerizable groups in the polymerizable liquid crystal compound is preferably 1 to 6, and more preferably 1 to 3.

Examples of the polymerizable liquid crystal compound include the compounds described in Makromol. Chem., (1989), Vol. 190, p. 2255, Advanced Materials (1993), Vol. 5, p. 107, U.S. Pat. Nos. 4,683,327A, 5,622,648A, 5,770,107A, WO95/22586A, WO95/24455A, WO97/00600A, WO98/23580A, WO98/52905A, JP1989-272551A (JP-H1-272551A), JP1994-16616A (JP-H6-16616A), JP1995-110469A (JP-H7-110469A), JP1999-80081A (JP-H11-80081A), JP2001-328973A, and the like. Two or more kinds of the polymerizable liquid crystal compounds may be used in combination. In a case where two or more kinds of the polymerizable liquid crystal compounds are used in combination, the alignment temperature can be lowered.

In addition, as a polymerizable liquid crystal compound other than the examples, for example, the cyclic organopolysiloxane compounds disclosed in JP1982-165480A (JP-S57-165480A) can be used. Further, as the above-described polymer liquid crystal compound, for example, a polymer in which a liquid crystal mesogenic group is introduced into the main chain, a side chain, or both the main chain and the side chain, a polymer cholesteric liquid crystal in which a cholesteryl group is introduced into a side chain, the liquid crystal polymers disclosed in JP1997-133810A (JP-H9-133810A), and the liquid crystal polymers disclosed in JP1999-293252A (JP-H11-293252A) can be used.

### —Disk-Like Liquid Crystal Compound—

As the disk-like liquid crystal compound, for example, the compounds described in JP2007-108732A or JP2010-244038A can be preferably used.

The amount of the polymerizable liquid crystal compound added to the liquid crystal composition based on the mass of solid contents (except for solvents) of the liquid crystal composition is preferably 75% to 99.9% by mass, more preferably 80% to 99% by mass, and still more preferably 85% to 90% by mass.

### —Surfactant—

The liquid crystal composition used for forming the liquid crystal layer may contain a surfactant.

It is preferable that the surfactant is a compound that can function as an alignment control agent contributing to the stable or rapid alignment of the liquid crystal compound 40 in the liquid crystal layer 102. Examples of the surfactant include a silicone-based surfactant and a fluorine-based surfactant, and preferred examples thereof include the fluorine-based surfactant.

Specific examples of the surfactant include the compounds described in paragraphs [0082] to [0090] of JP2014-119605A, the compounds described in paragraphs [0031] to [0034] of JP2012-203237A, the compounds exemplified in paragraphs [0092] and [0093] of JP2005-99248A, the compounds exemplified in paragraphs [0076] to [0078] and paragraphs [0082] to [0085] of JP2002-129162A, and the fluorine (meth)acrylate polymers described in paragraphs [0018] to [0043] of JP2007-272185A, and the like.

Furthermore, the surfactants may be used alone or in combination of two or more kinds thereof.

As the fluorine-based surfactant, the compounds described in paragraphs to of JP2014-119605A are preferable.

The addition amount of the surfactant in the liquid crystal composition is preferably 0.01% to 10% by mass, more preferably 0.01% to 5% by mass, and still more preferably 0.02% to 1% by mass with respect to the total mass of the liquid crystal compound.

### —Polymerization Initiator—

In a case where the liquid crystal composition includes a polymerizable compound, it is preferable that the liquid crystal composition contains a polymerization initiator. In an aspect where a polymerization reaction progresses with ultraviolet irradiation, it is preferable that the polymerization initiator is a photopolymerization initiator which initiates a polymerization reaction with ultraviolet irradiation.

Examples of the photopolymerization initiator include α-carbonyl compounds (described in the specifications of U.S. Pat. No. 2,367,661A and 2,367,670A), acyloin ether (described in the specification of U.S. Pat. No. 2,448,828A), α-hydrocarbon-substituted aromatic acyloin compounds (described in the specification of U.S. Pat. No. 2,722,512A), polynuclear quinone compounds (described in the specifications of U.S. Pat. Nos. 3,046,127A and 2,951,758A), combinations of triarylimidazole dimer and p-aminophenyl ketone (described in the specification of U.S. Pat. No. 3,549,367A), acridine compounds and phenazine compounds (described in JP1985-105667A (JP-S60-105667A) and the specification of U.S. Pat. No. 4,239,850A), and oxadiazole compounds (described in the specification of U.S. Pat. No. 4,212,970A).

A content of the photopolymerization initiator in the liquid crystal composition is preferably 0.1% to 20% by mass, and more preferably 0.5% to 12% by mass with respect to the content of the liquid crystal compound.

### —Crosslinking Agent—

In order to improve the film hardness after hardening and to improve durability, the liquid crystal composition may optionally include a crosslinking agent. As the crosslinking agent, a crosslinking agent that cures the liquid crystal composition with ultraviolet rays, heat, humidity, and the like can be suitably used.

The crosslinking agent is not particularly limited and can be appropriately selected depending on the purpose. Examples of the crosslinking agent include a polyfunctional acrylate compound such as trimethylol propane tri(meth)acrylate and pentaerythritol tri(meth)acrylate; an epoxy compound such as glycidyl (meth)acrylate and ethylene glycol diglycidyl ether; an aziridine compound such as 2,2-bis hydroxymethyl butanol-tris [3-(1-aziridinyl) propionate] and 4,4-bis(ethyleneiminocarbonylamino) diphenylmethane; an isocyanate compound such as hexamethylene diisocyanate and a biuret type isocyanate; a polyoxazoline compound having an oxazoline group at a side chain thereof; and an alkoxysilane compound such as vinyl trimethoxysilane and N-(2-aminoethyl)-3-aminopropyltrimethoxysilane. In addition, a known catalyst can be used depending on reactivity of the crosslinking agent, and in addition to improving the film hardness and the durability, productivity can be improved. These may be used alone or in combination of two or more kinds thereof.

The content of the crosslinking agent is preferably 3% to 20% by mass, and more preferably 5% to 15% by mass with respect to the solid content mass of the liquid crystal composition. In a case where the content of the crosslinking agent is in the range, an effect of improving a crosslinking density can be easily obtained and the stability of a liquid crystal phase is further improved.

### —Other Additives—

Optionally, a polymerization inhibitor, an antioxidant, an ultraviolet absorber, a light stabilizer, a coloring material, metal oxide fine particles, or the like can be added to the liquid crystal composition in a range where optical performance and the like do not deteriorate.

During the formation of an optically anisotropic layer, the liquid crystal composition is preferably used as a liquid.

The liquid crystal composition may include a solvent. The solvent is not limited and can be appropriately selected depending on the purpose, but an organic solvent is preferable.

The organic solvent is not limited and can be appropriately selected depending on the purpose. Examples of the organic solvent include ketones, alkyl halides, amides, sulfoxides, heterocyclic compounds, hydrocarbons, esters, and ethers. These may be used alone or in combination of two or more kinds thereof. Among these, the ketone is preferable in consideration of an environmental burden.

In addition, the liquid crystal diffraction element may have a layer other than the optically anisotropic layer, such as a support and an alignment film.

### (Support)

As a support that supports the alignment film and the optically anisotropic layer, various sheet-like materials (films or plate-like materials) can be used as long as they can support the alignment film and the optically anisotropic layer.

Furthermore, the support preferably has a transmittance of 50% or more, more preferably 70% or more, and still more preferably 85% or more with respect to diffracted light (near-infrared rays).

A thickness of the support is not limited and may be appropriately set depending on the use of the liquid crystal diffraction element, a material for forming the support, and the like in a range in which the alignment film and the optically anisotropic layer can be supported.

The thickness of the support is preferably 1 to 1,000 μm, more preferably 3 to 250 μm, and still more preferably 5 to 150 μm.

The support may be a single layer or a multi-layer.

In a case where the support is a single layer, examples thereof include supports formed of glass, triacetyl cellulose (TAC), polyethylene terephthalate (PET), polycarbonates, polyvinyl chloride, acryl, and polyolefin. In a case where the support is a multi-layer, examples thereof include a support that includes any of the above-described single-layered supports as a substrate; and has another layer provided on a surface of the substrate.

### (Alignment Film)

The alignment film is formed on a surface of the support.

The alignment film is an alignment film for aligning the liquid crystal compound 40 to a predetermined liquid crystal alignment pattern during the formation of the optically anisotropic layer.

As described above, in the present invention, the optically anisotropic layer has the liquid crystal alignment pattern in which the orientation of the optical axis 40A (refer to FIG. 7) derived from the liquid crystal compound 40 changes while continuously rotating along one in-plane direction. Accordingly, the alignment film is formed such that the optically anisotropic layer can form the liquid crystal alignment pattern.

In the following description, “the orientation of the optical axis 40A rotates” will also be simply referred to as “the optical axis 40A rotates”.

As the alignment film, various known films can be used.

Examples of the alignment film include a rubbed film formed of an organic compound such as a polymer, an obliquely deposited film formed of an inorganic compound, a film having a microgroove, and a film formed by lamination of Langmuir-Blodgett (LB) films formed with a Langmuir-Blodgett's method using an organic compound such as ω-tricosanoic acid, dioctadecylmethylammonium chloride, and methyl stearate.

The alignment film formed by a rubbing treatment can be formed by rubbing a surface of a polymer layer with paper or fabric in a predetermined direction multiple times.

As the material used for the alignment film, a material for forming polyimide, polyvinyl alcohol, a polymer having a polymerizable group described in JP1997-152509A (JP-H9-152509A), or an alignment film 32 and the like described in JP2005-97377A, JP2005-99228A, and JP2005-128503A is preferable.

The alignment film can be suitably used as a so-called photo-alignment film obtained by irradiating a photo-alignment material with polarized light or unpolarized light. That is, a photo-alignment film formed by applying a photo-alignment material to the support is suitably used as the alignment film.

The irradiation of polarized light can be performed in a direction orthogonal or oblique to the photo-alignment film, and the irradiation of unpolarized light can be performed in an oblique direction with respect to the photo-alignment film.

Preferable examples of the photo-alignment material used in the alignment film that can be used in the present invention include: an azo compound described in JP2006-285197A, JP2007-76839A, JP2007-138138A, JP2007-94071A, JP2007-121721A, JP2007-140465A, JP2007-156439A, JP2007-133184A, JP2009-109831A, JP3883848B, and JP4151746B; an aromatic ester compound described in JP2002-229039A; a maleimide- and/or alkenyl-substituted nadiimide compound having a photo-alignable unit described in JP2002-265541A and JP2002-317013A; a photocrosslinking silane derivative described in JP4205195B and JP4205198B, a photocrosslinking polyimide, a photocrosslinking polyamide, or a photocrosslinking polyester described in JP2003-520878A, JP2004-529220A, and JP4162850B; and a photodimerizable compound, in particular, a cinnamate compound, a chalcone compound, or a coumarin compound described in JP1997-118717A (JP-H9-118717A), JP1998-506420A (JP-H10-506420A), JP2003-505561A, WO2010/150748A, JP2013-177561A, and JP2014-12823A.

Among these, the azo compound, the photocrosslinking polyimide, the photocrosslinking polyamide, the photocrosslinking polyester, the cinnamate compound, or the chalcone compound is suitably used.

A thickness of the alignment film is not limited, and a thickness with which a required alignment function can be obtained may be appropriately set depending on the material for forming the alignment film.

The thickness of the alignment film is preferably 0.01 to 5 μm, and more preferably 0.05 to 2 μm.

A method for forming the alignment film is not limited, and various well-known methods corresponding to the material forming the alignment film can be used. Examples thereof include a method including: applying an alignment film to a surface of the support, drying the applied alignment film, and exposing the alignment film to laser light to form an alignment pattern.

FIG. 10 conceptually shows an example of an exposure device that exposes the alignment film to form an alignment pattern.

Δn exposure device 60 shown in FIG. 10 includes a light source 64 including a laser 62, a λ/2 plate 65 that changes a polarization direction of laser light M emitted from the laser 62, a beam splitter 68 that splits the laser light M emitted from the laser 62 into two rays MA and MB, mirrors 70A and 70B that are disposed on optical paths of the split two rays MA and MB, and λ/4 plates 72A and 72B.

Furthermore, the light source 64 emits linearly polarized light P₀. The λ/4 plate 72A converts the linearly polarized light P₀ (rays MA) into dextrorotatory circularly polarized light P_{R}, and the λ/4 plate 72B converts the linearly polarized light P₀ (rays MB) into levorotatory circularly polarized light P_{L}.

The support 30 having the alignment film 32 before an alignment pattern is formed thereon is disposed at an exposed area, the two rays MA and MB intersect and interfere with each other on the alignment film 32, and the alignment film 32 is irradiated with the interference light for exposure.

Due to the interference at this time, the polarization state of light with which the alignment film 32 is irradiated periodically changes according to interference fringes. This makes it possible to obtain an alignment film (hereinafter also referred to as a “patterned alignment film”) having an alignment pattern in which the alignment state changes periodically.

In the exposure device 60, the period of the alignment pattern can be adjusted by changing an intersection angle α between the two rays MA and MB. That is, by adjusting the intersecting angle α in the exposure device 60, in the alignment pattern in which the optical axis 40A derived from the liquid crystal compound 40 continuously rotates along one direction, the length of the single period over which the optical axis 40A rotates by 180° in the one direction in which the optical axis 40A rotates can be adjusted.

By forming the optically anisotropic layer on the alignment film 32 having such an alignment pattern in which the alignment state periodically changes, the optically anisotropic layer having the liquid crystal alignment pattern in which the optical axis 40A derived from the liquid crystal compound 40 continuously rotates along the one direction can be formed.

In addition, by rotating each of the optical axes of the λ/4 plates 72A and 72B by 90°, the rotation direction of the optical axis 40A can be reversed.

As described above, the patterned alignment film has an alignment pattern to obtain a liquid crystal alignment pattern in which the liquid crystal compound is aligned such that the orientation of the optical axis of the liquid crystal compound in the optically anisotropic layer formed on the patterned alignment film changes while continuously rotating along at least one in-plane direction. In a case where an axis directed along the orientation in which the liquid crystal compound is aligned is referred to as an alignment axis, it can be said that the patterned alignment film has an alignment pattern in which the orientation of the alignment axis changes while continuously rotating along at least one direction of in-plane directions. The alignment axis of the patterned alignment film can be detected by measuring absorption anisotropy. For example, in a case where the patterned alignment film is irradiated with linearly polarized light while rotating the patterned alignment film and the amount of light transmitted through the patterned alignment film is measured, the orientation with which the amount of light is maximum or minimum is observed by gradually changing along one in-plane direction.

Furthermore, in the present invention, the alignment film is provided as a preferred aspect and is not an essential configuration requirement.

For example, the following configuration can also be adopted, in which by forming an alignment pattern on a support using a method of subjecting the support to a rubbing treatment, a method of processing the support with laser light, or the like, the optically anisotropic layer has a liquid crystal alignment pattern in which the orientation of the optical axis 40A derived from the liquid crystal compound 40 changes while continuously rotating along at least one in-plane direction. That is, in the present invention, the support may be set to act as the alignment film.

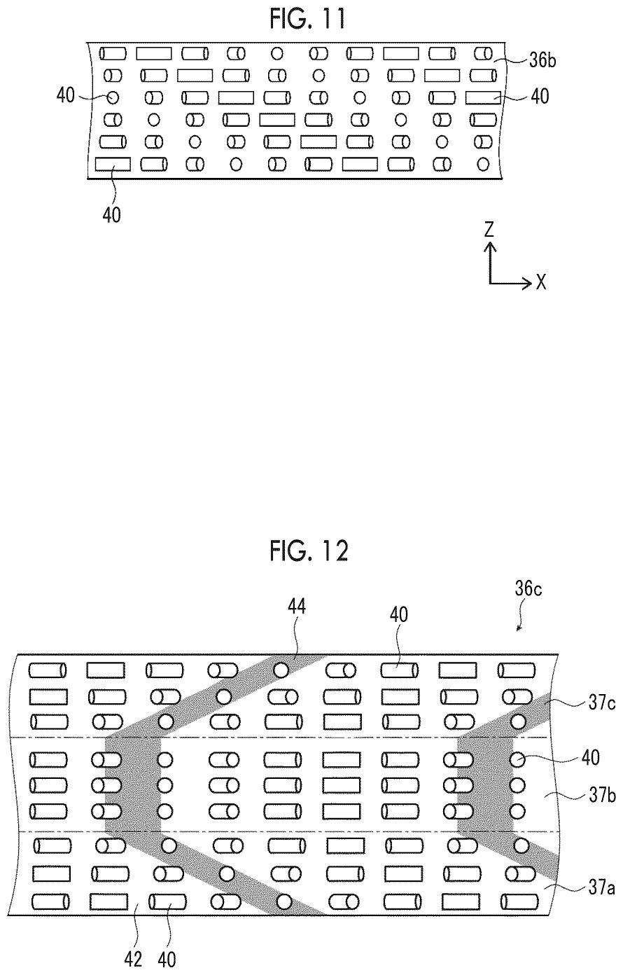

Here, in the optically anisotropic layer shown in FIGS. 6 and 7, the optical axes of the liquid crystal compounds arranged in the thickness direction are aligned in the same direction, but the present invention is not limited to this configuration. As in the optically anisotropic layer 36b shown in FIG. 11, a region in which the optical axis of the liquid crystal compound is twisted along the thickness direction may be provided in the plane. At this time, in the region having the twisted structure in the thickness direction, a twisted angle over the entire area in the thickness direction is 10° to 360°.

In this way, in a case where the optically anisotropic layer has the liquid crystal alignment pattern in which the orientation of the optical axis 40A changes while continuously rotating along the arrangement axis D in the plane and has the structure where the liquid crystal compound 40 is twisted in the thickness direction, in a cross section parallel to the arrangement axis D, a line segment that connects the liquid crystal compounds 40 facing the same direction in the thickness direction is tilted with respect to the main surface of the optically anisotropic layer. Furthermore, in an image obtained by observing a cross section of the optically anisotropic layer taken in the thickness direction along the arrangement axis D with a scanning electron microscope (SEM), a stripe pattern of bright portions and dark portions to be observed is tilted with respect to the main surface. This makes it possible to further improve the diffraction efficiency of the diffraction element.

In this way, in order for the optically anisotropic layer to have a configuration in which the liquid crystal compound is twisted and aligned in the thickness direction, the liquid crystal composition for forming an optically anisotropic layer may contain a chiral agent.

### —Chiral Agent (Optically Active Compound)—

The chiral agent has a function of inducing a helical structure of a liquid crystal phase. The chiral agent may be selected depending on the purposes since a helical twisted direction and a helical twisting power (HTP) to be induced vary depending on compounds.

The chiral agent is not particularly limited, and a well-known compound (for example, Liquid Crystal Device Handbook (No. 142 Committee of Japan Society for the Promotion of Science, 1989), Chapter 3, Article 4-3, chiral agent for twisted nematic (TN) or super twisted nematic (STN), p. 199), isosorbide, an isomannide derivative, or the like can be used.

The chiral agent generally includes an asymmetric carbon atom, but an axially asymmetric compound or a planar asymmetric compound, including no asymmetric carbon atom, can also be used as the chiral agent. Examples of the axially asymmetric compound or the planar asymmetric compound include binaphthyl, helicene, paracyclophane, and derivatives thereof. The chiral agent may also have a polymerizable group. In a case where both the chiral agent and the liquid crystal compound have a polymerizable group, a polymer having a repeating unit induced from the polymerizable liquid crystal compound and a repeating unit induced from the chiral agent can be formed by a polymerization reaction between the polymerizable chiral agent and the polymerizable liquid crystal compound. In this aspect, it is preferable that the polymerizable group in the polymerizable chiral agent is the same as the polymerizable group in the polymerizable liquid crystal compound. Accordingly, the polymerizable group of the chiral agent is preferably an unsaturated polymerizable group, an epoxy group, or an aziridinyl group, more preferably an unsaturated polymerizable group, and still more preferably an ethylenically unsaturated polymerizable group.

In addition, the chiral agent may be a liquid crystal compound.

In a case where the chiral agent has a photoisomerization group, a desired twisted alignment corresponding to a luminescence wavelength can be formed by irradiation of an actinic ray or the like through a photomask after coating and alignment, which is preferable. As the photoisomerization group, an isomerization site of a compound exhibiting photochromic properties, an azo group, an azoxy group, or a cinnamoyl group is preferable. As specific compounds, the compounds described in JP2002-80478A, JP2002-80851A, JP2002-179668A, JP2002-179669A, JP2002-179670A, JP2002-179681A, JP2002-179682A, JP2002-338575A, JP2002-338668A, JP2003-313189A, JP2003-313292A, or the like can be used.

The content of the chiral agent in the liquid crystal composition is preferably 0.01% to 200% by mole, and more preferably 1% to 30% by mole with respect to the molar content of the liquid crystal compound.

In addition, the optically anisotropic layer may be configured to have regions in which twisted states (twisted angles and twisted directions) are different in the thickness direction. In a case of such a configuration, in a cross sectional image obtained by observing a cross section of the optically anisotropic layer taken in the thickness direction along the one direction in which the orientation of the optical axis of the liquid crystal compound changes while continuously rotating with a scanning electron microscope, a bright portion and a dark portion extending from one main surface to another main surface are observed, and the dark portion has one or two or more inflection points of angle.

FIG. 12 shows an example of such an optically anisotropic layer. Furthermore, in FIG. 12, bright portions 42 and dark portions 44 are shown to overlap a cross section of an optically anisotropic layer 36c. In the following description, the image obtained by observing the cross section taken in the thickness direction along the one direction in which the optical axis rotates with an SEM will also be simply referred to as a “cross sectional SEM image”.

In the cross sectional SEM image of the optically anisotropic layer 36c shown in FIG. 12, the dark portion 44 has two inflection points at which the angle changes. That is, the optically anisotropic layer 36c can also have three regions including a region 37a, a region 37b, and a region 37c corresponding to the inflection points of the dark portion 44 in the thickness direction.

The optically anisotropic layer 36c also has, at any position in the thickness direction, the liquid crystal alignment pattern in which the optical axis derived from the liquid crystal compound 40 rotates clockwise to the left direction in the drawing in the in-plane direction in a view from the top in the drawing. In addition, the single period of the liquid crystal alignment pattern is fixed in the thickness direction.

In addition, as shown in FIG. 12, in the lower region 37a in the thickness direction, the liquid crystal compound 40 is twisted and aligned to be helically twisted clockwise (to the right) from the upper side to the lower side in the drawing in the thickness direction.

In the middle region 37b in the thickness direction, the liquid crystal compound 40 is not twisted in the thickness direction, and the optical axes of the liquid crystal compounds 40 laminated in the thickness direction face the same direction. That is, the optical axes of the liquid crystal compounds 40 present at the same position in the in-plane direction face the same direction.

In the upper region 37c in the thickness direction, the liquid crystal compound 40 is twisted and aligned to be helically twisted counterclockwise (to the left) from the upper side to the lower side in the drawing in the thickness direction.

That is, in the region 37a, the region 37b, and the region 37c of the optically anisotropic layer 36c shown in FIG. 12, the twisted states of the liquid crystal compounds 40 in the thickness direction are different from each other.

In the optically anisotropic layer having the liquid crystal alignment pattern in which the optical axis derived from the liquid crystal compound continuously rotates in the one direction, the bright portions and the dark portions in the cross sectional SEM image of the optically anisotropic layer are observed to connect the liquid crystal compounds facing the same orientation.

For example, in FIG. 12, the dark portions 44 are observed to connect the liquid crystal compounds 40 of which the optical axes face a direction orthogonal to the paper plane.

In the lowermost region 37a in the thickness direction, the dark portion 44 is tilted to the upper left side in the drawing. In the middle region 37b, the dark portion 44 extends in the thickness direction. In the uppermost region 37c, the dark portion 44 is tilted to the upper right side in the drawing.

That is, the optically anisotropic layer 36c shown in FIG. 12 has two inflection points of angle where the angle of the dark portion 44 changes. In addition, in the uppermost region 37c, the dark portion 44 is tilted to the upper right side. In the lowermost region 37b, the dark portion 44 is tilted to the upper left side. That is, in the region 37c and the region 37a, the tilt directions of the dark portions 44 are different from each other.

Further, the optically anisotropic layer 36c shown in FIG. 12 has one inflection point at which the dark portion 44 is folded in a direction opposite to the tilt direction.

Specifically, regarding the dark portion 44 of the optically anisotropic layer 36c, the tilt direction in the region 37c and the tilt direction in the region 37b are opposite to each other. Therefore, at the inflection point positioned at the interface between the region 37c and the region 37b, the tilt direction is folded in the opposite direction. That is, the optically anisotropic layer 36c has one inflection point at which the tilt direction is folded in the opposite direction.

In addition, in the region 37c and the region 37a of the optically anisotropic layer 36c, for example, the thicknesses are the same, and the twisted states of the liquid crystal compounds 40 in the thickness direction are different from each other. Therefore, as shown in FIG. 12, the bright portions 42 and the dark portions 44 in the cross sectional SEM image are formed in a substantially C-shape.

Accordingly, in the optically anisotropic layer 36c, the shape of the dark portion 44 is symmetrical with respect to the center line in the thickness direction.

In such an optically anisotropic layer 36c, that is, in the optically anisotropic layer 36c in which the cross sectional SEM image has the bright portions 42 and the dark portions 44 extending from one surface to another surface and each of the dark portions 44 has one or two or more inflection points of angle, the wavelength dependence of the diffraction efficiency can be reduced, and light can be diffracted with the same diffraction efficiency irrespective of wavelengths. In addition, the wide angle characteristics of the optically anisotropic layer 36c are improved, and thus, the light can be diffracted with the same diffraction efficiency regardless of the incidence angle.

Furthermore, in the example shown in FIG. 12, the dark portion 44 is configured to have two inflection points of angle. However, the present invention is not limited to this configuration, and the dark portion 44 may have one inflection point of angle or may have three or more inflection points of angle. For example, in the configuration in which the dark portion 44 of the optically anisotropic layer has one inflection point of angle, for example, the optically anisotropic layer may consist of the region 37c and the region 37a shown in FIG. 12, may consist of the region 37c and the region 37b, or may consist of the region 37b and the region 37a. Alternatively, in a case where the dark portion 44 of the optically anisotropic layer is configured to have three inflection points of angle, the region 37c and the region 37a shown in FIG. 12 may be alternately provided two by two.

The single period Λ in the optically anisotropic layer may be appropriately set depending on the refraction angle of transmission rays. The single period Λ is preferably about 1 to 3 times the wavelength of the near-infrared rays emitted from the light source unit. By setting the single period Λ to be in this range, the refraction angle can be set to oblique incidence and emission angles as shown by a and B in FIG. 1.

Hereinbefore, the blood flow measurement device of the embodiment of the present invention has been described in detail, but the present invention is not limited to the above-described examples and various improvements and changes can be made without departing from the spirit of the present invention.

### EXPLANATION OF REFERENCES

30: support
   32: alignment film
   36, 36b and 36c: liquid crystal diffraction element (optically anisotropic layer)
   37a to 37c: region
   60: exposure device
   62: laser
   64: light source
   65: λ/2 plate
   68: beam splitter
   70A, 70B: mirror
   72A, 72B: λ/4 plate
   100, 100a to 100d: blood flow measurement device
   102: control unit
   104: light source unit
   106, 106a to 106d: first polarizing element
   108: light receiving section
   110, 110a to 106d: second polarizing element
   112: housing
   120, 122: linear polarizer
   120a, 122a: first linear polarizer
   120b, 122b: second linear polarizer
   124, 125: λ/4 plate
   126, 127: retardation layer
   128: liquid crystal diffraction element
   S: living body
   R: region
   Λ: single period
   D: arrangement axis
   L₁, L₄: incidence rays
   L₂, L₅: transmission rays
   M: laser light
   MA, MB: rays
   P_{O}: linearly polarized light
   P_{R}: dextrorotatory circularly polarized light
   P_{L}: levorotatory circularly polarized light
   α: intersecting angle

What is claimed is:

1. A blood flow measurement device comprising:
a light source unit that irradiates an object with near-infrared rays; and
a light receiving section that receives scattered light generated by scattering of the near-infrared rays emitted from the light source unit by the object, the blood flow measurement device further comprising:
a first polarizing element that is disposed on a front surface of the light source unit, includes a layer formed of a liquid crystal compound, and changes a polarization state of the near-infrared rays; and
a second polarizing element that is disposed on a front surface of the light receiving section, includes a layer formed of a liquid crystal compound, and changes a polarization state of the near-infrared rays.

2. The blood flow measurement device according to claim 1,
wherein the layer formed of the liquid crystal compound included in the first polarizing element is a linear polarizer.

3. The blood flow measurement device according to claim 2,
wherein the first polarizing element further includes a λ/4 plate.

4. The blood flow measurement device according to claim 3,
wherein the λ/4 plate exhibits reverse wavelength dispersibility.

5. The blood flow measurement device according to claim 1,
wherein the first polarizing element has a first linear polarizer, a retardation layer, and a second linear polarizer in this order, and
at least one of the first linear polarizer or the second linear polarizer is the layer formed of the liquid crystal compound.

6. The blood flow measurement device according to claim 5,
wherein the retardation layer exhibits reverse wavelength dispersibility.

7. The blood flow measurement device according to claim 1,
wherein the layer formed of the liquid crystal compound included in the first polarizing element has a liquid crystal alignment pattern in which an orientation of an optical axis derived from the liquid crystal compound changes while continuously rotating along at least one in-plane direction.

8. The blood flow measurement device according to claim 1,
wherein the liquid crystal compound is a rod-like liquid crystal compound or a disk-like liquid crystal compound.

9. The blood flow measurement device according to claim 2,
wherein the layer formed of the liquid crystal compound included in the first polarizing element has a liquid crystal alignment pattern in which an orientation of an optical axis derived from the liquid crystal compound changes while continuously rotating along at least one in-plane direction.

10. The blood flow measurement device according to claim 2,
wherein the liquid crystal compound is a rod-like liquid crystal compound or a disk-like liquid crystal compound.

11. The blood flow measurement device according to claim 3,
wherein the layer formed of the liquid crystal compound included in the first polarizing element has a liquid crystal alignment pattern in which an orientation of an optical axis derived from the liquid crystal compound changes while continuously rotating along at least one in-plane direction.

12. The blood flow measurement device according to claim 3,
wherein the liquid crystal compound is a rod-like liquid crystal compound or a disk-like liquid crystal compound.

13. The blood flow measurement device according to claim 4,
wherein the layer formed of the liquid crystal compound included in the first polarizing element has a liquid crystal alignment pattern in which an orientation of an optical axis derived from the liquid crystal compound changes while continuously rotating along at least one in-plane direction.

14. The blood flow measurement device according to claim 4,
wherein the liquid crystal compound is a rod-like liquid crystal compound or a disk-like liquid crystal compound.

15. The blood flow measurement device according to claim 5,
wherein the layer formed of the liquid crystal compound included in the first polarizing element has a liquid crystal alignment pattern in which an orientation of an optical axis derived from the liquid crystal compound changes while continuously rotating along at least one in-plane direction.

16. The blood flow measurement device according to claim 5,
wherein the liquid crystal compound is a rod-like liquid crystal compound or a disk-like liquid crystal compound.

17. The blood flow measurement device according to claim 6,
wherein the layer formed of the liquid crystal compound included in the first polarizing element has a liquid crystal alignment pattern in which an orientation of an optical axis derived from the liquid crystal compound changes while continuously rotating along at least one in-plane direction.

18. The blood flow measurement device according to claim 6,
wherein the liquid crystal compound is a rod-like liquid crystal compound or a disk-like liquid crystal compound.

19. The blood flow measurement device according to claim 7,
wherein the liquid crystal compound is a rod-like liquid crystal compound or a disk-like liquid crystal compound.
